(19) United States
(12) Patent Application Publication
(10) Pub. No.: US 2025/0000365 A1
(43) Pub. Date: Jan. 2, 2025

(54) Wearable Device with a Circular or Polygonal Array of Light Emitters and Light Receivers to Measure Biometric Parameters

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 18/885,728

(22) Filed: Sep. 15, 2024

Related U.S. Application Data
(63) Continuation-in-part of application No. 18/775,128, filed on Jul. 17, 2024.
(63) Continuation-in-part of application No. 18/617,950, filed on Mar. 27, 2024.
(63) Continuation-in-part of application No. 18/121,841, filed on Mar. 15, 2023.
Application No. 18/775,128 is a continuation-in-part of application No. 18/617,950, filed on Mar. 27, 2024.
Application No. 18/617,950 is a continuation-in-part of application No. 18/121,841, filed on Mar. 15, 2023.
Application No. 18/121,841 is a continuation-in-part of application No. 18/121,841, filed on Mar. 15, 2023.
Application No. 18/121,841 is a continuation-in-part of application No. 17/903,746, filed on Sep. 6, 2022, now abandoned.
Application No. 17/903,746 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/294,746, filed on Oct. 16, 2016, now Pat. No. 10,627,861.
Application No. 15/294,746 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/418,620, filed on Jan. 27, 2017, now abandoned.
Application No. 15/418,620 is a continuation-in-part of application No. 15/944,746, filed on Apr. 3, 2018, now abandoned.
Application No. 15/963,061 is a continuation-in-part of application No. 14/992,073, filed on Jan. 11, 2016, now abandoned.
Application No. 14/992,073 is a continuation-in-part of application No. 14/550,953, filed on Nov. 22, 2014, now abandoned.
Application No. 14/550,953 is a continuation-in-part of application No. 15/431,769, filed on Feb. 14, 2017, now abandoned.
Application No. 15/431,769 is a continuation-in-part of application No. 14/951,475, filed on Nov. 24, 2015, now Pat. No. 10,314,492.
Application No. 14/951,475 is a continuation-in-part of application No. 15/294,746, filed on Oct. 16, 2016, now Pat. No. 10,627,861.
Application No. 15/294,746 is a continuation-in-part of application No. 15/206,215, filed on Jul. 8, 2016, now abandoned.
Application No. 15/206,215 is a continuation-in-part of application No. 14/992,073, filed on Jan. 11, 2016, now abandoned.
Application No. 14/992,073 is a continuation-in-part of application No. 14/330,649, filed on Jul. 14, 2014, now abandoned.
Application No. 14/330,649 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/948,308, filed on Nov. 21, 2015, now abandoned.
Application No. 14/992,073 is a continuation-in-part of application No. 14/562,719, filed on Dec. 7, 2014, now Pat. No. 10,130,277.
Application No. 14/562,719 is a continuation-in-part of application No. 13/616,238, filed on Sep. 14, 2012, now abandoned.
Application No. 14/951,475 is a continuation-in-part of application No. 14/071,112, filed on Nov. 4, 2013, now abandoned.
Application No. 14/071,112 is a continuation-in-part of application No. 13/901,131, filed on May 23, 2013, now Pat. No. 9,536,449.
Application No. 14/948,308 is a continuation-in-part of application No. 14/550,953, filed on Nov. 22, 2014, now abandoned.
Application No. 14/550,953 is a continuation-in-part of application No. 14/449,387, filed on Aug. 1, 2014, now abandoned.
Application No. 14/449,387 is a continuation-in-part of application No. 14/132,292, filed on Dec. 18, 2013, now Pat. No. 9,442,100.
Application No. 14/132,292 is a continuation-in-part of application No. 13/901,099, filed on May 23, 2013, now Pat. No. 9,254,099.
Application No. 14/330,649 is a continuation-in-part of application No. 13/523,739, filed on Jun. 14, 2012, now Pat. No. 9,042,596.
(60) Provisional application No. 63/542,077, filed on Oct. 2, 2023.
(60) Provisional application No. 63/542,077, filed on Oct. 2, 2023.
(60) Provisional application No. 63/279,773, filed on Nov. 16, 2021.
(60) Provisional application No. 63/171,838, filed on Apr. 7, 2021.
(60) Provisional application No. 62/930,013, filed on Nov. 4, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/800,478, filed on Feb. 2, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/549,587, filed on Aug. 24, 2017.
(60) Provisional application No. 62/439,147, filed on Dec. 26, 2016.
(60) Provisional application No. 62/439,147, filed on Dec. 26, 2016.
(60) Provisional application No. 62/349,277, filed on Jun. 13, 2016.
(60) Provisional application No. 62/311,462, filed on Mar. 22, 2016.
(60) Provisional application No. 62/297,827, filed on Feb. 20, 2016.
(60) Provisional application No. 62/349,277, filed on Jun. 13, 2016.
(60) Provisional application No. 62/245,311, filed on Oct. 23, 2015.
(60) Provisional application No. 62/349,277, filed on Jun. 13, 2016.
(60) Provisional application No. 61/932,517, filed on Jan. 28, 2014.

(51) Int. Cl. A61B 5/00

(52) U.S. Cl. CPC A61B 5/0075; A61B 5/681; A61B 2562/043; A61B 2562/046

(57) ABSTRACT

A wearable device for measuring biometric parameters includes a circular or polygonal array of sets of light emitters and light receivers. Light transmitted through body tissue is received and analyzed to measure one or more biometric parameters such as oxygenation level, heart rate, heart rate variability, blood pressure, hydration level, and/or blood glucose level. Each set of light emitters includes light emitters which emit light of different colors. This device can also include one or more compressible opaque light barriers which surround light receivers.

### CROSS-REFERENCE TO RELATED APPLICATIONS

This application is a continuation-in-part of U.S. patent application Ser. No. 18/775,128 filed on 2024 Jul. 17. This application is a continuation-in-part of U.S. patent application Ser. No. 18/617,950 filed on 2024 Mar. 27. This application claims the priority benefit of U.S. provisional application 63/542,077 filed on 2023 Oct. 2. This application is a continuation-in-part of U.S. patent application Ser. No. 18/121,841 filed on 2023 Mar. 15.

U.S. patent application Ser. No. 18/775,128 was a continuation-in-part of U.S. patent application Ser. No. 18/617,950 filed on 2024 Mar. 27. U.S. patent application Ser. No. 18/775,128 was a continuation-in-part of U.S. patent application Ser. No. 18/121,841 filed on 2023 Mar. 15.

U.S. patent application Ser. No. 18/617,950 claimed the priority benefit of U.S. provisional application 63/542,077 filed on 2023 Oct. 2. U.S. patent application Ser. No. 18/617,950 was a continuation-in-part of U.S. patent application Ser. No. 18/121,841 filed on 2023 Mar. 15.

U.S. patent application Ser. No. 18/121,841 was a continuation-in-part of U.S. patent application Ser. No. 17/903,746 filed on 2022 Sep. 6. U.S. patent application Ser. No. 18/121,841 was a continuation-in-part of U.S. patent application Ser. No. 17/239,960 filed on 2021 Apr. 26. U.S. patent application Ser. No. 18/121,841 was a continuation-in-part of U.S. patent application Ser. No. 16/737,052 filed on 2020 Jan. 8.

U.S. patent application Ser. No. 17/903,746 was a continuation-in-part of U.S. patent application Ser. No. 16/568,580 filed on 2019 Sep. 12. U.S. patent application Ser. No. 17/903,746 was a continuation-in-part of U.S. patent application Ser. No. 16/737,052 filed on 2020 Jan. 8. U.S. patent application Ser. No. 17/903,746 was a continuation-in-part of U.S. patent application Ser. No. 17/239,960 filed on 2021 Apr. 26. U.S. patent application Ser. No. 17/903,746 claimed the priority benefit of U.S. provisional application 63/279,773 filed on 2021 Nov. 16.

U.S. patent application Ser. No. 17/239,960 claimed the priority benefit of U.S. provisional application 63/171,838 filed on 2021 Apr. 7. U.S. patent application Ser. No. 17/239,960 was a continuation-in-part of U.S. patent application Ser. No. 16/737,052 filed on 2020 Jan. 8.

U.S. patent application Ser. No. 16/737,052 claimed the priority benefit of U.S. provisional application 62/930,013 filed on 2019 Nov. 4. U.S. patent application Ser. No. 16/737,052 claimed the priority benefit of U.S. provisional application 62/857,942 filed on 2019 Jun. 6. U.S. patent application Ser. No. 16/737,052 claimed the priority benefit of U.S. provisional application 62/814,713 filed on 2019 Mar. 6. U.S. patent application Ser. No. 16/737,052 claimed the priority benefit of U.S. provisional application 62/814,692 filed on 2019 Mar. 6. U.S. patent application Ser. No. 16/737,052 claimed the priority benefit of U.S. provisional application 62/800,478 filed on 2019 Feb. 2. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25 which issued as U.S. Pat. No. 10,772,559 on 2020 Sep. 15. U.S. patent application Ser. No. 16/737,052 was a continuation-in-part of U.S. patent application Ser. No. 15/725,330 filed on 2017 Oct. 5 which issued as U.S. Pat. No. 10,607,507 on 2020 Mar. 31. U.S. patent application Ser. No. 16/737,052 was a continuation-in-part of U.S. patent application Ser. No. 15/431,769 filed on 2017 Feb. 14. U.S. patent application Ser. No. 16/737,052 was a continuation-in-part of U.S. patent application Ser. No. 15/294,746 filed on 2016 Oct. 16 which issued as U.S. Pat. No. 10,627,861 on 2020 Apr. 21.

U.S. patent application Ser. No. 16/568,580 claimed the priority benefit of U.S. provisional application 62/857,942 filed on 2019 Jun. 6. U.S. patent application Ser. No. 16/568,580 claimed the priority benefit of U.S. provisional application 62/814,713 filed on 2019 Mar. 6. U.S. patent application Ser. No. 16/568,580 claimed the priority benefit of U.S. provisional application 62/814,692 filed on 2019 Mar. 6. U.S. patent application Ser. No. 16/568,580 was a continuation-in-part of U.S. patent application Ser. No. 15/963,061 filed on 2018 Apr. 25 which issued as U.S. Pat. No. 10,772,559 on 2020 Sep. 15. U.S. patent application Ser. No. 16/568,580 was a continuation-in-part of U.S. patent application Ser. No. 15/725,330 filed on 2017 Oct. 5 which issued as U.S. Pat. No. 10,607,507 on 2020 Mar. 31. U.S. patent application Ser. No. 16/568,580 was a continuation-in-part of U.S. patent application Ser. No. 15/431,769 filed on 2017 Feb. 14. U.S. patent application Ser. No. 16/568,580 was a continuation-in-part of U.S. patent application Ser. No. 15/418,620 filed on 2017 Jan. 27. U.S. patent application Ser. No. 16/568,580 was a continuation-in-part of U.S. patent application Ser. No. 15/294,746 filed on 2016 Oct. 16 which issued as U.S. Pat. No. 10,627,861 on 2020 Apr. 21.

U.S. patent application Ser. No. 15/963,061 was a continuation-in-part of U.S. patent application Ser. No. 14/992,073 filed on 2016 Jan. 11. U.S. patent application Ser. No. 15/963,061 was a continuation-in-part of U.S. patent application Ser. No. 14/550,953 filed on 2014 Nov. 22.

U.S. patent application Ser. No. 15/725,330 claimed the priority benefit of U.S. provisional application 62/549,587 filed on 2017 Aug. 24. U.S. patent application Ser. No. 15/725,330 claimed the priority benefit of U.S. provisional application 62/439,147 filed on 2016 Dec. 26. U.S. patent application Ser. No. 15/725,330 was a continuation-in-part of U.S. patent application Ser. No. 15/431,769 filed on 2017 Feb. 14. U.S. patent application Ser. No. 15/725,330 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/431,769 claimed the priority benefit of U.S. provisional application 62/439,147 filed on 2016 Dec. 26. U.S. patent application Ser. No. 15/431,769 claimed the priority benefit of U.S. provisional application 62/349,277 filed on 2016 Jun. 13. U.S. patent application Ser. No. 15/431,769 claimed the priority benefit of U.S. provisional application 62/311,462 filed on 2016 Mar. 22. U.S. patent application Ser. No. 15/431,769 was a continuation-in-part of U.S. patent application Ser. No. 15/294,746 filed on 2016 Oct. 16 which issued as U.S. Pat. No. 10,627,861 on 2020 Apr. 21. U.S. patent application Ser. No. 15/431,769 was a continuation-in-part of U.S. patent application Ser. No. 15/206,215 filed on 2016 Jul. 8. U.S. patent application Ser. No. 15/431,769 was a continuation-in-part of U.S. patent application Ser. No. 14/992,073 filed on 2016 Jan. 11. U.S. patent application Ser. No. 15/431,769 was a continuation-in-part of U.S. patent application Ser. No. 14/330,649 filed on 2014 Jul. 14.

U.S. patent application Ser. No. 15/418,620 claimed the priority benefit of U.S. provisional application 62/297,827 filed on 2016 Feb. 20. U.S. patent application Ser. No. 15/418,620 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/294,746 claimed the priority benefit of U.S. provisional application 62/349,277 filed on 2016 Jun. 13. U.S. patent application Ser. No. 15/294,746 claimed the priority benefit of U.S. provisional application 62/245,311 filed on 2015 Oct. 23. U.S. patent application Ser. No. 15/294,746 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/206,215 claimed the priority benefit of U.S. provisional application 62/349,277 filed on 2016 Jun. 13. U.S. patent application Ser. No. 15/206,215 was a continuation-in-part of U.S. patent application Ser. No. 14/951,475 filed on 2015 Nov. 24 which issued as U.S. Pat. No. 10,314,492 on 2019 Jun. 11. U.S. patent application Ser. No. 15/206,215 was a continuation-in-part of U.S. patent application Ser. No. 14/948,308 filed on 2015 Nov. 21.

U.S. patent application Ser. No. 14/992,073 was a continuation-in-part of U.S. patent application Ser. No. 14/562,719 filed on 2014 Dec. 7 which issued as U.S. Pat. No. 10,130,277 on 2018 Nov. 20. U.S. patent application Ser. No. 14/992,073 was a continuation-in-part of U.S. patent application Ser. No. 13/616,238 filed on 2012 Sep. 14.

U.S. patent application Ser. No. 14/951,475 was a continuation-in-part of U.S. patent application Ser. No. 14/071,112 filed on 2013 Nov. 4. U.S. patent application Ser. No. 14/951,475 was a continuation-in-part of U.S. patent application Ser. No. 13/901,131 filed on 2013 May 23 which issued as U.S. Pat. No. 9,536,449 on 2017 Jan. 3.

U.S. patent application Ser. No. 14/948,308 was a continuation-in-part of U.S. patent application Ser. No. 14/550,953 filed on 2014 Nov. 22. U.S. patent application Ser. No. 14/948,308 was a continuation-in-part of U.S. patent application Ser. No. 14/449,387 filed on 2014 Aug. 1. U.S. patent application Ser. No. 14/948,308 was a continuation-in-part of U.S. patent application Ser. No. 14/132,292 filed on 2013 Dec. 18 which issued as U.S. Pat. No. 9,442,100 on 2016 Sep. 13. U.S. patent application Ser. No. 14/948,308 was a continuation-in-part of U.S. patent application Ser. No. 13/901,099 filed on 2013 May 23 which issued as U.S. Pat. No. 9,254,099 on 2016 Feb. 9.

U.S. patent application Ser. No. 14/562,719 claimed the priority benefit of U.S. provisional application 61/932,517 filed on 2014 Jan. 28.

U.S. patent application Ser. No. 14/330,649 was a continuation-in-part of U.S. patent application Ser. No. 13/523,739 filed on 2012 Jun. 14 which issued as U.S. Pat. No. 9,042,596 on 2015 May 26.

The entire contents of these applications are incorporated herein by reference.

### FEDERALLY SPONSORED RESEARCH

Not Applicable

### SEQUENCE LISTING OR PROGRAM

Not Applicable

### BACKGROUND—FIELD OF INVENTION

This invention relates to wearable devices for measuring biometric parameters.

### INTRODUCTION

There are many potential health-related benefits from noninvasive measurement of biometric parameters such as body oxygenation level, heart rate, heart rate variability, blood pressure, body hydration level, and blood glucose level. Considerable progress has been made in recent years toward measuring some of these parameters by incorporating spectroscopic sensors (e.g. light emitters and light receivers) into wearable devices such as smart watches. However, challenges remain. For example, a wearable device can shift as a person moves. This can change the location of a light emitter relative to a person's body and also cause gaps between the device and the person's body. Light can pass through these gaps and reach a light receiver directly, without being transmitted through body tissue as intended.

### REVIEW OF THE RELEVANT ART

U.S. patent application 20190142313 (Abou Ismail et al., May 16, 2019, “System and Method for Non-Invasive Continuous Real-Time Blood Glucose Monitoring”) discloses using an impedance sensor and permittivity to measure blood glucose. U.S. patent application 20190159703 (Aggarwal et al., May 30, 2019, “System and Method for Obtaining Blood Glucose Concentration Using Temporal Independent Component Analysis (ICA)”) discloses using near infrared spectroscopy (NIR) to measure blood glucose. U.S. patent application 20160146726 (Aggarwal, May 26, 2016, “Wearable Device for Detection of Contaminants and Method Thereof”) discloses a wearable spectrometer for analyzing the chemical composition of substances.

U.S. patent application 20190154584 (Ahn et al., May 23, 2019, “Spectroscopy Apparatus, Spectroscopy Method, and Bio-Signal Measuring Apparatus”) discloses a spectroscopy apparatus with a dispersive element which divides an incident light into a plurality of lights having different output angles. U.S. patent application 20190117140 (Al-Ali et al., Apr. 25, 2019, “Advanced Pulse Oximetry Sensor”) discloses a pulse oximetry sensor with a light diffuser and light concentrator. U.S. patent application 20190216322 (Anikanov et al., Jul. 18, 2019, “Compact Spectrometer System for Non-Invasive Measurement of Absorption and Transmission Spectra in Biological Tissue Samples”) discloses a spectrometer for analyzing an inhomogeneous scattering medium.

U.S. patent application 20240125915 (Au et al., Apr. 18, 2024, “Method, Apparatus, and System for Wireless Sensing Measurement and Reporting”) and U.S. patent application 20240179550 (Au et al., May 30, 2024, “Method, Apparatus, and System for Wireless Sensing Measurement and Reporting”) disclose methods, devices, and systems for wireless sensing including transmitting a time series of at least one wireless sounding signal (WSS). U.S. patent application 20220091683 (Beyhs et al., Mar. 24, 2022, “Ring Input Device with Pressure-Sensitive Input”) and U.S. Pat. No. 11,733,790 (Beyhs et al., Aug. 22, 2023, “Ring Input Device with Pressure-Sensitive Input”) disclose a ring with a pressure-sensitive input mechanism. U.S. patent application 20230359291 (Beyhs et al., Nov. 9, 2023, “Ring Input Device with Variable Rotational Resistance”) discloses a ring input device with variable rotational resistance mechanisms which change the rotational friction of a rotating outer band.

U.S. patent application 20180143150 (Bezemer et al., May 24, 2018, “Apparatus and Methods That Use Magnetic Induction Spectroscopy to Monitor Tissue Fluid Content”) discloses using magnetic induction spectroscopy to measure tissue fluid. U.S. patent application 20240058686 (Bhandarkar et al., Feb. 22, 2024, “Smart Wearable Device”) discloses a smart ring with an electronics unit that is selectively attachable to a coupling mount. U.S. patent application 20240188881 (Bonificio et al., Jun. 13, 2024, “Wearable Ring Device and Method of Monitoring Sleep Apnea Events”) discloses a finger-worn band with a pulse oximetry sensor on an inner surface of the band. U.S. patent application 20240065631 (Brooks, Feb. 29, 2024, “Pressure Adjustment for Biometric Measurement”) discloses a user device with a pressure sensor to determine whether the pressure between a user's body and the device is within a proper range.

U.S. patent application 20180271448 (Bynam et al., Sep. 27, 2018, “Method of Enabling Feature Extraction for Glucose Monitoring Using Near-Infrared (NIR) Spectroscopy”) discloses using near-infrared (NIR) spectroscopy to measure blood glucose. U.S. patent application 20230008487 (Caizzone et al., Jan. 12, 2023, “System and Method for Smart Rings Employing Sensor Spatial Diversity”) discloses a ring for photoplethysmographic sensing which uses sensor spatial diversity to enhance the quality and the reliability of measurements. U.S. Pat. No. 10,401,800 (Cardinali et al., Sep. 3, 2019, “Indicators for Wearable Electronic Devices”) discloses a wearable device with a biometric sensor and illuminated biometric status indicator.

U.S. patent application 20230118067 (Chang et al., Apr. 20, 2023, “Electronic Device and Method to Measure Bioelectrical Impedance”) discloses an electronic device with a plurality of electrodes, a sensor connected to the electrodes, a memory, and a processor which obtains contact impedances through the sensor. U.S. patent application 20190069843 (Chatterjee et al., Mar. 7, 2019, “Wearable Personal Information System”) discloses a wearable optical device with a shield to block ambient light. U.S. patent application 20140058226 (Chernobro et al., Feb. 27, 2014, “Method and Apparatus for In Vivo Optical Measurement of Blood Glucose Concentration”) discloses using differential scattering spectroscopy and Doppler microscopy to measure blood glucose.

U.S. patent application 20190049296 (Cho et al., Feb. 14, 2019, “Light Filter and Spectrometer Including the Light Filter”) discloses a spectrometer with different spectrum modulation portions. U.S. patent application 20190167190 (Choi et al., Jun. 6, 2019, “Healthcare Apparatus and Operating Method Thereof”) discloses a healthcare apparatus with a plurality of light sources which emit light of different wavelengths, a light detector, and a processor configured to obtain a blood glucose level. U.S. patent application 20230200744 (Choi et al., Jun. 29, 2023, “Apparatus and Method for Estimating Target Component”) discloses an apparatus for estimating a target component via a spectrometer.

U.S. patent application 20240049350 (Choi et al., Feb. 8, 2024, “Electronic Apparatus and Operating Method Thereof”) discloses an electronic which can receive a requests for wireless connection from a first device, identify a usage of a wireless bandwidth of the first device, and determine whether a wireless connection to the first device is possible based on a remaining wireless bandwidth of the electronic device. U.S. patent application 20240048675 (Choi et al., Feb. 8, 2024, “Electronic Device and Operation Method Thereof”) discloses a device with processors which obtain a rotation angle of the device and determine whether the rotation angle is greater than or equal to a reference rotation angle.

U.S. patent application 20190013368 (Chung et al., Jan. 10, 2019, “Near-Infrared Light Organic Sensors, Embedded Organic Light Emitting Diode Panels, and Display Devices Including the Same”) discloses an OLED panel which is embedded with a near-infrared organic photosensor, wherein this structure enables biometric recognition. U.S. patent application 20230350503 (D'Amone et al., Nov. 2, 2023, “Ring Input Devices”) and U.S. Pat. No. 11,714,494 (D'Amone et al., Aug. 1, 2023, “Ring Input Devices”) disclose how a head-mountable device can be operated with a ring input device worn on a finger of a user. U.S. patent application 20180103883 (Darty et al., Apr. 19, 2018, “Systems and Methods for Measuring Tissue Oxygenation”) discloses using images with different spectral bands to measure tissue oxygenation.

U.S. Pat. No. 8,868,149 (Eisen et al., Oct. 21, 2014, “Photoplethysmography Device and Method”) and U.S. Pat. No. 9,149,216 (Eisen et al., Oct. 6, 2015, “Photoplethysmography Device and Method”) and U.S. patent applications 20110082355 (Eisen et al., Apr. 7, 2011, “Photoplethysmography Device and Method”), 20130131475 (Eisen et al., May 23, 2013, “Photoplethysmography Device and Method”), and 20150105638 (Eisen et al., Apr. 16, 2015, “Photoplethysmography Device and Method”) disclose using photoplethysmography and dynamic light scattering for biometric measurement. U.S. Pat. No. 9,314,197 (Eisen et al., Apr. 19, 2016, “Wearable Pulse Oximetry Device”) and U.S. patent application 20140200423 (Eisen et al., Jul. 17, 2014, “Wearable Pulse Oximetry Device”) disclose a wrist-worn pulse oximetry device which is worn over the ulna.

U.S. Pat. No. 9,730,622 (Eisen et al., Aug. 15, 2017, “Wearable Pulse Oximetry Device”) and U.S. patent application 20160278676 (Eisen et al., Sep. 29, 2016, “Wearable Pulse Oximetry Device”) disclose a wearable pulse oximetry device with two light emitters with two different wavelengths. U.S. patent application 20230218192 (Eom et al., Jul. 13, 2023, “Wrist-Type Body Component Measuring Apparatus and Body Component Measuring Method Using the Same”) discloses a wrist-worn band with: a first input electrode and a first output electrode disposed on an inside surface of the band; and a second input electrode and a second output electrode disposed on an outside surface of the band. U.S. patent application 20240138721 (Eom et al., May 2, 2024, “Apparatus and Method for Estimating Concentration of Analyte Component”) discloses an apparatus for estimating a component level using a plurality of light sources with different central wavelengths and at least one light detector.

U.S. Pat. No. 11,829,831 (Ershov et al., Nov. 28, 2023, “Electronic System with Ring Device”) discloses a wearable electronic device with a coil which is formed from metal traces. U.S. patent application 20240000380 (Fei et al., Jan. 4, 2024, “Wearable Device”) discloses an annular case with an energy storage unit, an information transmission unit, and an optical identification assembly. U.S. patent application 20180333107 (Garcia Sada et al., Nov. 22, 2018, “Non-Invasive Wearable Device, Process and Systems with Adjustable Operation”) discloses a wearable device with a flexible housing and an array of sensors. U.S. Pat. No. 11,660,228 (Goff et al., May 30, 2023, “Positional Obstructive Sleep Apnea Detection System”) discloses an obstructive sleep apnea detection device which uses an optical engagement surface adapted to engage a user's skin.

U.S. patent applications 20220085841 (Gretarsson et al., Mar. 17, 2022, “Smart Ring”) and 20220407550 (Gretarsson et al., Dec. 22, 2022, “Smart Ring”) disclose a wearable device which detects inputs, gestures, and/or biometric parameters. U.S. patent application 20240146350 (Gretarsson et al., May 2, 2024, “Smart Ring”) discloses a ring, band, or necklace with a pressure-sensitive mechanism that receives user input in the form of applied pressure. U.S. patent application 20180325431 (Guarin et al., Nov. 15, 2018, “Electromagnetic Wave Sensor for Determining a Hydration Status of a Body Tissue In Vivo”) discloses using an electromagnetic wave sensor to measure body tissue hydration.

U.S. Pat. No. 10,317,200 (Han et al., Jun. 11, 2019, “Multi-Mode Sensor for Surface Orientation”) discloses a wearable device with an orientation sensor and multiple pairings between light emitters and light detectors. U.S. Pat. No. 10,893,833 (Haverinen et al., Jan. 19, 2021, “Wearable Electronic Device and Method for Manufacturing Thereof”) discloses a wearable electronic device made from non-ceramic material. U.S. patent application 20190216340 (Holz et al., Jul. 18, 2019, “Sensor Device”) discloses a multi-dimensional optical sensor for biometric measurements. U.S. patent application 20210289897 (Hsu et al., Sep. 23, 2021, “Smart Ring”) discloses a smart ring with an antenna chip and a metal ring which functions as an antenna. U.S. Pat. No. 11,911,181 (Huttunen et al., Feb. 27, 2024, “Flexible Wearable Ring Device”) discloses a wearable device made from flexible materials.

U.S. Pat. No. 8,725,226 (Isaacson, May 13, 2014, “Optical Sensor Path Selection”) discloses a device with multiple possible pairings of light emitters and detectors to scan different tissue depths for biometric measurement. U.S. Pat. No. 9,498,158 (Isaacson, Nov. 22, 2016, “Optical Sensor Path Selection”) discloses a device with multiple light emitters to scan different tissue depths for biometric measurement. U.S. patent application 20170196493 (Isaacson, Jul. 13, 2017, “Optical Sensor Path Selection”) discloses a device with multiple optical elements and detectors to measure a biometric parameter. U.S. patent applications 20230056434 (Jang et al., Feb. 23, 2023, “Apparatus and Method for Estimating Blood Pressure”) and 20230070636 (Kang et al., Mar. 9, 2023, “Apparatus and Method for Estimating Blood Pressure”) disclose an apparatus for estimating blood pressure using a pulse wave sensor.

U.S. patent applications 20230309844 (Jang et al., Oct. 5, 2023, “Apparatus and Method for Estimating Blood Pressure”) and 20240172945 (Park et al., May 30, 2024, “Apparatus and Method for Estimating Blood Pressure”) disclose an apparatus and method for estimating blood pressure using a photoplethysmogram (PPG) sensor. U.S. patent application 20240115212 (Jang et al., Apr. 11, 2024, “Apparatus and Method for Estimating Physiological Variables”) discloses an apparatus for estimating physiological variables using sensors and a neural-network-based physiological variable estimation model. U.S. patent application 20190252569 (Jo et al., Aug. 15, 2019, “Near-Infrared Light Sensors Including 2-Dimensional Insulator”) discloses a near infrared light sensor with a 2D material semiconductor layer on a substrate.

U.S. patent application 20180140238 (Johnson et al., May 24, 2018, “Regional Oximetry Sleeve for Mobile Device”) discloses a mobile computing device with a sleeve. U.S. patent application 20230301540 (Jung et al., Sep. 28, 2023, “Apparatus and Method of Measuring Bio Signal”) discloses a method of measuring a biosignal by positioning electrodes and switching an impedance measurer. U.S. patent application 20230053252 (Jung, Feb. 16, 2023, “Electronic Device Adjusting Oxygen Saturation and Method for Controlling the Same”) discloses a device with a first sensor which detects movement and a second sensor which measures oxygen saturation.

U.S. patent application 20190133469 (Just et al., May 9, 2019, “Physiological Monitoring Devices Having Sensing Elements Decoupled from Body Motion”) discloses a biometric measuring device with multiple bands to reduce motion artifacts. U.S. patent application 20220409072 (Kang et al., Dec. 29, 2022, “Apparatus and Method for Estimating Bio-Information”) discloses an apparatus to estimate biometric parameters using a pulse wave sensor with channels in an isotropic shape. U.S. patent application 20230007884 (Kang et al., Jan. 12, 2023, “Apparatus and Method for Estimating Bio-Information”) discloses an apparatus to estimate biometric parameters which measures pulse wave signals from an object.

U.S. Pat. No. 11,864,871 (Kang et al., Jan. 9, 2024, “Wearable Device and Method of Measuring Bio-Signal”) discloses an external light collector, an auxiliary light source, and a light receiver. U.S. patent application 20230205325 (Khan, Jun. 29, 2023, “Wearable Apparatus and Control Method Thereof”) discloses a wearable apparatus with a display, a strap, at least one sensor configured to acquire posture information, and at least one processor. U.S. patent application 20160077587 (Kienzle et al., Mar. 17, 2016, “Smart Ring”) discloses a smart ring with at least one flexion sensor which detects the distance between the sensor and a finger segment. U.S. Pat. No. 7,371,217 (Kim et al., May 13, 2008, “Device for the Non-Invasive Measurement of Blood Glucose Concentration by Millimeter Waves and Method Thereof”) discloses using millimeter waves to measure glucose level.

U.S. patent application 20190033217 (Kim, Jan. 31, 2019, “Spectrum Measurement Apparatus and Spectrum Measurement Method”) discloses a spectrum measurement apparatus with a plurality of light sources which emit light at different wavelengths, a light detector, and a processor. U.S. patent application 20190150746 (Kim, May 23, 2019, “Bio-Information Measuring Apparatus and Bio-Information Measuring Method”) discloses a biometric spectroscopy device with a pressure sensor. U.S. Pat. No. 10,842,429 (Kinnunen et al., Nov. 24, 2020, “Method and System for Assessing a Readiness Score of a User”) discloses a method and a system for assessing a user's readiness based on their movements. U.S. Pat. No. 11,793,454 (Kinnunen et al., Oct. 24, 2023, “Method and System for Providing Feedback to User for Improving Performance Level Management Thereof”) discloses a method of collecting a set of user information and determining a user's current performance.

U.S. Pat. No. 11,986,313 (Kinnunen et al., May 21, 2024, “Method and System for Monitoring and Improving Sleep Pattern of User”) discloses methods and systems for providing feedback to a user for adjusting their sleep pattern. U.S. patent application 20170231566 (Klimek et al., Aug. 17, 2017, “Tissue Interface”) discloses a garment with a sensor module for biometric measurement. U.S. patent application 20180055449 (Ko et al., Mar. 1, 2018, “Wearable Measurement Apparatus”) discloses a wearable biometric device with elastic portions. U.S. patent application 20210204815 (Koskela et al., Jul. 8, 2021, “An Optical Sensor System of a Wearable Device, A Method for Controlling Operation of an Optical Sensor System and Corresponding Computer Program Product”) discloses a wearable optical sensor system including at least two photo transmitters, a photoreceiver, receiving electronics, and a microcontroller.

U.S. Pat. No. 11,980,439 (Koskela et al., May 14, 2024, “Optical Sensor System of a Wearable Device, A Method for Controlling Operation of an Optical Sensor System and Corresponding Computer Program Product”) discloses a system comprising at least two photo transmitters, a photoreceiver, receiving electronics, and a microcontroller. U.S. patent application 20230225671 (Kosman et al., Jul. 20, 2023, “Wearable Health Apparatus for the Collection of Wellness Data and Providing Feedback Therefrom to the Wearer”) discloses a ring with replaceable outer shells, as well as hardware and software that allow the user to communicate with a cell phone, cloud provider, table, personal computer or AI assistant. U.S. patent application 20180317786 (Kulach et al., Nov. 8, 2018, “Pulse Spectroscopy”) discloses a wearable photoplethysmogram (PPG) sensor.

U.S. patent application 20160206251 (Kwon et al., Jul. 21, 2016, “Apparatus for Detecting Bio-Information”) and U.S. Pat. No. 10,349,847 (Kwon et al., Jul. 6, 2019. “Apparatus for Detecting Bio-Information”) disclose an apparatus with a light-emitting diode (LED), a laser diode (LD), and an optical detector. U.S. Pat. No. 10,349,847 (Kwon et al., Jul. 6, 2019, “Apparatus for Detecting Bio-Information”) and U.S. patent application 20160206251 (Kwon et al., Jul. 21, 2016, “Apparatus for Detecting Bio-Information”) disclose using a two-dimensional optical array to measure biometric parameters. U.S. patent application 20240188834 (Kwon et al., Jun. 13, 2024, “Apparatus and Method for Measuring Blood Pressure”) discloses an apparatus for estimating blood pressure using a pulse wave sensor.

U.S. patent application 20180220906 (LeBoeuf et al., Aug. 9, 2018, “Physiological Monitoring Apparatus and Networks”) discloses ear-worn devices for biometric and environmental monitoring. U.S. patent application 20190025120 (Lee et al., Jan. 24, 2019, “Spectrometer and Spectrum Measurement Method Utilizing Same”) discloses a spectrometer with a first unit spectral filter which absorbs or reflects light in a part of a wavelength band of a light spectrum of an incident target, a second unit spectral filter which absorbs or reflects light in a wavelength band different from the part of the wavelength band, a first light detector configured to detect a first light spectrum passing through the first unit spectral filter, a second light detector configured to detect a second light spectrum passing through the second unit spectral filter, and a processing unit.

U.S. patent application 20190113387 (Lee et al., Apr. 18, 2019, “Spectrometric Sensor Control Method and Electronic Device for Supporting Same”) discloses spectroscopic sensor with multiple wavelength bands. U.S. patent application 20220386885 (Lee et al., Dec. 8, 2022, “Wearable Electronic Device Measuring Blood Pressure and Method for Operating The Same”) discloses a wearable electronic device with a memory, a first sensor, a second sensor, and a processor. U.S. patent application 20230000405 (Lee et al., Jan. 5, 2023, “Apparatus and Method for Estimating Bio-Information Based on Bio-Impedance”) discloses an apparatus to estimate biometric parameters using an impedance sensor, including a pair of input electrodes and a pair of receiving electrodes.

U.S. patent application 20230157645 (Lee et al., May 25, 2023, “Apparatus and Method for Estimating Bio-Information”) discloses an apparatus to estimate biometric parameters using a spectrometer. U.S. patent application 20240045473 (Lee et al., Feb. 8, 2024, “Electronic Device and Method for Operating Electronic Device”) discloses an electronic device with movable housings, at least one sensor, and one or more electromagnets. U.S. patent application 20230324293 (Lee, Oct. 12, 2023, “Apparatus and Method for Estimating Body Water Status”) discloses an apparatus for estimating body hydration level with a near-infrared light spectrometer. U.S. patent application 20190120689 (Leem et al., Apr. 25, 2019, “Combination Sensors and Electronic Devices”) discloses a biometric device with stacked infrared sensors.

U.S. Pat. No. 12,007,727 (Leith et al., Jun. 11, 2024, “Watch Band with Fit Detection”) discloses a watch band with an adjustable capacitor whose capacitance changes when the watch band configuration changes. U.S. patent application 20190155385 (Lim et al., May 23, 2019, “Smart Ring Providing Multi-Mode Control in a Personal Area Network”) discloses a smart ring which provides multi-mode control in a personal area network. U.S. patent application 20240046505 (Liu et al., Feb. 8, 2024, “Electronic Device and Method with Pose Prediction”) discloses an electronic device for predicting a pose and a method for operating the electronic device. U.S. Pat. No. 11,188,160 (Liu, Nov. 30, 2021, “Wireless Controlling System Implemented with Smart Ring and Wireless Controlling Method Thereof”) a wireless controlling system including a smart ring and an identification program installed in a mobile device.

U.S. patent application 20170224263 (Lobbestael et al., Aug. 10, 2017, “Tissue Site Detection”) discloses an optical biometric sensor which identifies a particular tissue site. U.S. patent application 20160350581 (Manuel et al., Dec. 1, 2016, “Smart Ring with Biometric Sensor”) discloses a ring comprising a ring body and a biometric sensor. U.S. Pat. No. 11,850,069 (Mars et al., Dec. 26, 2023, “Wearable Device and Methods of Manufacturing”) discloses a smart ring with a battery, a PCB, a fingerprint sensor, a temperature sensor, a memory, and a processing unit. U.S. Pat. No. 11,902,791 (Mars et al., Feb. 13, 2024, “Reader Device with Sensor Streaming Data and Methods”) discloses an access control system with a controller having an antenna interface to broadcast identifying data.

U.S. Pat. No. 11,916,900 (Mars et al., Feb. 27, 2024, “Authorized Remote Control Device Gesture Control Methods and Apparatus”) discloses a method for controlling a remote control device which includes capturing biometric data. U.S. patent application 20240187407 (Mars et al., Jun. 6, 2024, “Methods and Apparatus for Facilitating NFC Transactions”) discloses a method for controlling a remote control device with a session token in response to an authentication request. U.S. patent application 20190246977 (Miller et al., Aug. 15, 2019, “Optical Sensor for Wearable Devices”) discloses methods, systems, apparatuses, and/or devices which emit light into a body, receive light from a depth below a surface of the body, and determine a physiological condition of the body.

U.S. patent application 20190200883 (Moon et al., Jul. 4, 2019, “Bio-Signal Measuring Apparatus and Operating Method Thereof”) discloses a bio-signal measuring apparatus with a photodetector and an array of light sources around the photodetector. U.S. patent application 20230233084 (Moon et al., Jul. 27, 2023, “Method and Apparatus for Correcting Error of Optical Sensor, and Apparatus for Estimating Biometric Information”) discloses a method of correcting an optical sensor error by adjusting the brightness of a light source. U.S. patent application 20210072833 (Mutlu et al., Mar. 11, 2021, “Self-Mixing Interferometry-Based Gesture Input System Including a Wearable or Handheld Device”) discloses a device with one or more SMI sensors which emit beams of electromagnetic radiation, wherein each beam is emitted in a different direction.

U.S. Pat. No. 9,642,578 (Newberry, May 9, 2017, “System and Method for Health Monitoring Using a Non-Invasive, Multi-Band Biosensor”) and U.S. Pat. No. 9,980,676 (Newberry, May 29, 2018, “System and Method for Health Monitoring Using a Non-Invasive, Multi-Band Biosensor”) disclose a PPG sensor which uses multiple wavelengths. U.S. Pat. No. 10,321,860 (Newberry, Jun. 18, 2019, “System and Method for Glucose Monitoring”) discloses a glucose biosensor with optical fibers. U.S. patent application 20210089126 (Nickerson, Mar. 25, 2021, “Smart Ring”) discloses a smart ring with a capacitive touch sensor. U.S. patent application 20230350492 (Nickerson, Nov. 2, 2023, “Smart Ring”) discloses a smart ring worn which is controlled based on its position.

U.S. patent application 20230085555 (Nomvar et al., Mar. 16, 2023, “A Non-Invasive Continuous Blood Glucose Monitor”) discloses a non-invasive device for measuring glucose levels. U.S. patent application 20240112563 (Norman et al., Apr. 4, 2024, “Bluetooth Enabled Smart Ring”) discloses a smart ring device with wireless communication with a computing device that transitions between one or more states based on user and/or device inputs. U.S. patent application 20230154033 (Oh et al., May 18, 2023, “Method and Device for Estimating Poses and Models of Object”) discloses a method for object pose and model estimation via acquiring a global feature of an input image.

U.S. patent application 20230190118 (Park et al., Jun. 22, 2023, “Apparatus and Method for Estimating Blood Pressure”) discloses an apparatus for estimating blood pressure which extracts a cardiac output feature, a first candidate total peripheral resistance feature, and a second candidate peripheral resistance feature. U.S. patent application 20240081663 (Park et al., Mar. 14, 2024, “Apparatus for Estimating Bio-Information and Method of Detecting Abnormal Bio-Signal”) discloses an apparatus with a photoplethysmogram (PPG) sensor. U.S. patent application 20160292563 (Park, Oct. 6, 2016, “Smart Ring”) discloses systems and methods for pairing a smart ring with a primary device.

U.S. patent application 20190104942 (Peru et al., Apr. 11, 2019, “Spectroscopic System and Method Therefor”) discloses probe which analyzes saliva for biometric measurement. U.S. Pat. No. 11,666,230 (Piccinini et al., Jun. 6, 2023, “Electronic Device and Method for Noninvasive, Continuous Blood Pressure Monitoring”) discloses an electronic device and method for continuous noninvasive blood pressure monitoring. U.S. patent application 20170325742 (Prior et al., Nov. 16, 2017, “Universal Fingertip Sensor”) discloses a finger-tip-worn optical biometric sensor device. U.S. Pat. No. 11,895,383 (Prushinskiy et al., Feb. 6, 2024, “Electronic Device Including Optical Sensor”) discloses an electronic device with a housing which is rotatably arranged and an optical sensor assembly.

U.S. patent application 20240012479 (Qiu et al., Jan. 11, 2024, “Ring Enabling Its Wearer to Enter Control Commands”) discloses systems and methods, including a smart ring, which enable a user to control electronic devices in a local network. U.S. patent application 20180140237 (Rajan et al., May 24, 2018, “Device and Method for Determining Biological Indicator Levels in Tissue”) discloses using at least two light emitters to measure a biological parameter. U.S. Pat. No. 11,925,441 (Rantanen et al., Mar. 12, 2024, “Techniques for Determining Blood Pressure Based on Morphological Features of Pulses Preliminary Class”) discloses a wearable device with one or more light emitting components, one or more photodetectors, and a controller that couples the light emitting components to the photodetectors.

U.S. patent application 20170311823 (Rausch et al., Nov. 2, 2017, “Optical Trigger for Measurement”) discloses detecting and using feature in a photoplethysmogram signal for biometric measurement. U.S. patent application 20240000387 (Realubit et al., Jan. 4, 2024, “Finger Wearable Health Monitoring Device”) discloses a finger-worn health monitoring device comprising a circular metal shell. U.S. patent applications 20220334639 (Sanchez, Oct. 20, 2022, “Projection System for Smart Ring Visual Output”) and U.S. patent application 20220383741 (Sanchez, Dec. 1, 2022, “Non-Visual Outputs for a Smart Ring”), and U.S. Pat. No. 11,462,107 (Sanchez, Oct. 4, 2022, “Light Emitting Diodes and Diode Arrays for Smart Ring Visual Output”), disclose a smart ring system for displaying information concerning driving conditions.

U.S. Pat. No. 11,479,258 (Sanchez, Oct. 25, 2022, “Smart Ring System for Monitoring UVB Exposure Levels and Using Machine Learning Technique to Predict High Risk Driving Behavior”) discloses systems and methods determine a driver's fitness to safely operate a moving vehicle based on UVB exposure. U.S. patent application 20230072436 (Sanchez, Mar. 9, 2023, “Harvesting Energy for a Smart Ring Via Piezoelectric Charging”) discloses a smart ring which harvests mechanical energy using piezoelectricity. U.S. patent applications 20230143293 (Sanchez, May 11, 2023, “Biometric Authentication Using a Smart Ring”) and U.S. patent application 20230153416 (Sanchez, May 18, 2023, “Proximity Authentication Using a Smart Ring”) discloses systems and methods for performing biometric authentication using a smart ring.

U.S. patent application 20230174114 (Sanchez, Jun. 8, 2023, “Smart Ring System for Measuring Stress Levels and Using Machine Learning Techniques to Predict High Risk Driving Behavior”) discloses systems and methods determine a driver's fitness to safely operate a moving vehicle based on their stress level. U.S. patent application 20230205170 (Sanchez, Jun. 29, 2023, “Soft Smart Ring and Method of Manufacture”) discloses a smart ring with a body made from flexible material, a first part, a second part removably connected to the first part, and at least one pair of break-away portions disposed within the body. U.S. patent application 20230361588 (Sanchez, Nov. 9, 2023, “Smart Ring Power and Charging”) discloses a smart ring with a both a removable power source and an internal power source.

U.S. Pat. No. 11,949,673 (Sanchez, Apr. 2, 2024, “Gesture Authentication Using a Smart Ring”) discloses systems and methods for multi-factor authentication using a smart ring. U.S. Pat. No. 11,937,905 (Singleton et al., Mar. 26, 2024, “Techniques for Leveraging Data Collected by Wearable Devices and Additional Devices”) discloses a method comprising receiving physiological data from a wearable device and environmental data from an external device. U.S. patent application 20160262673 (Skorich et al., Sep. 15, 2016, “Segmented Sensor”) discloses an optical biometric sensor with a planar substrate. U.S. patent application 20180070850 (Stafford et al., Mar. 15, 2018, “Apparatus and Method for Detecting Body Composition and Correlating It With Cognitive Efficiency”) discloses a biometric device to measure body hydration and correlate it with cognitive efficiency.

U.S. Pat. No. 11,275,453 (Tham et al., Mar. 15, 2022, “Smart Ring for Manipulating Virtual Objects Displayed By a Wearable Device”) discloses systems, devices, media, and methods for using a ring to manipulate a virtual object displayed by smart eyewear. U.S. Pat. No. 11,580,300 (Tham et al., Feb. 14, 2023, “Ring Motion Capture and Message Composition System”) discloses systems, devices, media, and methods for composing and sharing a message based on the motion of a ring. U.S. patent application 20230021838 (Tse et al., Jan. 26, 2023, “Wearable Electronic Device”) discloses a wearable electronic device with conductive areas on both inner and outer surfaces. U.S. patent application 20190167170 (Varsavsky et al., Jun. 6, 2019, “Methods and Systems for Improving the Reliability of Orthogonally Redundant Sensors”) discloses using orthogonally redundant sensors to measure glucose level.

U.S. Pat. No. 12,076,142 (Venugopal et al., Sep. 3, 2024, “Physiological Monitoring System for Measuring Oxygen Saturation”) discloses a wearable device with a housing having a plurality of windows through which light emitters emit light and through which light detectors receive light. U.S. Pat. No. 10,444,834 (Vescovi, Oct. 15, 2019, “Devices, Methods, and User Interfaces for a Wearable Electronic Ring Computing Device”) discloses an electronic device with a finger-ring-mounted touchscreen. U.S. patent application 20150220109 (von Badinski et al., Aug. 6, 2015, “Wearable Computing Device”) and U.S. Pat. No. 9,582,034 (von Badinski et al., Feb. 28, 2017, “Wearable Computing Device”) disclose a finger ring comprising an interior wall, an exterior wall, a flexible circuit board, and a window that facilitates data transmission, battery recharge, and/or status indication.

U.S. patent application 20170235933 (von Badinski et al., Aug. 17, 2017, “Wearable Computing Device”) and U.S. Pat. No. 10,156,867 (von Badinski et al., Dec. 18, 2018, “Wearable Computing Device”) disclose a method for using a finger ring to identify an authorized user by illuminating a portion of the user's skin, imaging the portion, and then generating a capillary map. U.S. Pat. No. 10,768,666 (von Badinski et al., Sep. 8, 2020, “Wearable Computing Device”) discloses a smart ring which unlocks a client computing device, wherein the ring includes an accelerometer, a gyroscope, and/or other motion sensor. U.S. Pat. No. 11,188,124 (von Badinski et al., Nov. 30, 2021, “Wearable Computing Device”) discloses a smart ring with a curved housing having a U-shape interior, a motion sensor, a memory, a transceiver, a temperature sensor, and a processor.

U.S. Pat. No. 11,599,147 (von Badinski et al., Mar. 7, 2023, “Wearable Computing Device”) discloses a smart ring with a curved housing having a U-shape interior, a semi-flexible PCB, a motion sensor, a memory, a transceiver, a temperature sensor, and a processor. U.S. patent application 20230213970 (von Badinski et al., Jul. 6, 2023, “Wearable Computing Device”) discloses a smart ring with a body having an inner surface and an outer surface, wherein a cavity is formed on the inner surface of the body part and an electronic part is arranged in the cavity. U.S. patent application 20230213970 (von Badinski et al., Jul. 6, 2023, “Wearable Computing Device”) discloses a smart ring with a curved housing having a U-shape interior, a semi-flexible PCB, a motion sensor, a memory, a transceiver, a temperature sensor, and a processor.

U.S. patent applications 20230376072 (von Badinski et al., Nov. 23, 2023, “Wearable Computing Device”) and 20230384827 (von Badinski et al., Nov. 30, 2023, “Wearable Computing Device”) disclose a smart ring with a curved housing having a U-shape interior, a curved battery, a semi-flexible PCB, a motion sensor, a memory, a transceiver, a temperature sensor, and a processor. U.S. patent application 20230376071 (von Badinski et al., Nov. 23, 2023, “Wearable Computing Device”) discloses a smart ring comprising an external housing component with an outer circumferential surface and an inner circumferential surface, wherein a portion of the inner circumferential surface contacts a person's finger. U.S. patent application 20230409080 (von Badinski et al., Dec. 21, 2023, “Wearable Computing Device”) discloses a smart ring with a curved housing with a substantially transparent portion, a curved battery, a semi-flexible PCB, a motion sensor, a memory, a transceiver, a temperature sensor, and a processor.

U.S. Pat. No. 11,868,178 (von Badinski et al., Jan. 9, 2024, “Wearable Computing Device”) discloses a smart ring with a curved housing having a U-shape interior, a battery, a semi-flexible PCB, a motion sensor, a memory, a transceiver, a temperature sensor, and a processor. U.S. Pat. No. 11,868,179 (von Badinski et al., Jan. 9, 2024, “Wearable Computing Device”) discloses a smart ring with a curved housing having a U-shape interior, a battery, a semi-flexible PCB, a motion sensor, a memory, a transceiver, a temperature sensor, a processor, an infrared light emitter, and a visible light emitter. U.S. Pat. No. 11,874,701 (von Badinski et al., Jan. 16, 2024, “Wearable Computing Device”) discloses a smart ring with a curved housing having a U-shape interior, a battery, a semi-flexible PCB, a motion sensor, a memory, a transceiver, a temperature sensor, and a processor which identifies gestures based on data from the motion sensor.

U.S. Pat. No. 11,874,702 (von Badinski et al., Jan. 16, 2024, “Wearable Computing Device”) discloses a smart ring with a curved housing with a substantially transparent portion, a battery, a semi-flexible PCB, a motion sensor, a memory, a transceiver, a temperature sensor, and a processor. U.S. patent application 20240126328 (von Badinski et al., Apr. 18, 2024, “Wearable Computing Device”) discloses a smart ring with a curved housing having a U-shape interior, a battery, a semi-flexible PCB, a galvanic sensor, light emitters, light receivers, a memory, a transceiver, a temperature sensor, and a processor. U.S. patent application 20240126329 (von Badinski et al., Apr. 18, 2024, “Wearable Computing Device”) discloses a wearable ring device with a ring-shaped housing, a printed circuit board, and a sensor module with infrared light emitters, visible light emitters, and light receivers.

U.S. patent application 20240126330 (von Badinski et al., Apr. 18, 2024, “Wearable Computing Device”) discloses a wearable ring device with a ring-shaped housing comprising two metallic materials, a printed circuit board, light emitters, and light receivers. U.S. patent application 20240134417 (von Badinski et al., Apr. 25, 2024, “Wearable Computing Device”) discloses a wearable ring device with a ring-shaped housing, a thermoelectric generator, a printed circuit board, light emitters, and light receivers. U.S. patent application 20240143027 (von Badinski et al., May 2, 2024, “Wearable Computing Device”) discloses a wearable ring device with a ring-shaped housing with one or more windows, a printed circuit board, light emitters, and light receivers. U.S. patent application 20240143028 (von Badinski et al., May 2, 2024, “Wearable Computing Device”) discloses a wearable ring device with a ring-shaped housing, a printed circuit board, and a sensor module that includes red light emitters, infrared light emitters, and light receivers.

U.S. patent application 20240168521 (von Badinski et al., May 23, 2024, “Wearable Computing Device”) discloses a wearable ring device with a ring-shaped housing, one or more temperature sensors, a printed circuit board, light emitters, and light receivers. U.S. Pat. No. 12,013,725 (von Badinski et al., Jun. 18, 2024, “Wearable Computing Device”) discloses a wearable ring device with a ring-shaped housing, a printed circuit board, a haptic feedback module, red light emitters, infrared light emitters, and light receivers. U.S. patent application 20240201736 (von Badinski et al., Jun. 20, 2024, “Wearable Computing Device”) discloses a wearable ring device with a ring-shaped housing, a first conductive contact component, a second conductive contact component, a printed circuit board, light emitters, and light receivers.

U.S. patent application 20170209095 (Wagner et al., Jul. 27, 2017, “Optical Physiological Sensor Modules with Reduced Signal Noise”) discloses an optical sensor module with light guides which have outwardly-diverging axial directions. U.S. patent application 20180020979 (Wagner et al., Jan. 25, 2018, “Optical Adapters for Wearable Monitoring Devices”) discloses a wearable optical biometric sensor with stabilizing members. U.S. patent application 20180042554 (Wagner et al., Feb. 15, 2018, “Optical Monitoring Apparatus and Methods”) discloses a biometric device with a digital camera and a photoplethysmography (PPG) sensor. U.S. patent application 20180078209 (Wagner et al., Mar. 22, 2018, “Stabilized Sensor Modules and Monitoring Devices Incorporating Same”) discloses a wearable optical biometric sensor with stabilizing protrusions.

U.S. patent application 20230043018 (Wai et al., Feb. 9, 2023, “Smart Ring for Use with a User Device and Wi-Fi Network”) discloses a smart ring with a battery, a memory, processing circuitry, a plurality of sensors, and a plurality of antennas. U.S. patent applications 20180074010 (Wang et al., Mar. 15, 2018, “Application of Electrochemical Impedance Spectroscopy in Sensor Systems, Devices, and Related Methods” and 20180074011 (Wang et al., Mar. 15, 2018, “Application of Electrochemical Impedance Spectroscopy in Sensor Systems, Devices, and Related Methods”), and 20180074012 (Wang et al., Mar. 15, 2018, “Application of Electrochemical Impedance Spectroscopy in Sensor Systems, Devices, and Related Methods”) disclose using electrochemical impedance spectroscopy to measure glucose level. U.S. Pat. No. 10,739,820 (Wang et al., Aug. 11, 2020, “Expandable Ring Device”) discloses a ring device including force sensors, ultrasonic sensors, inertial measurement units, optical sensors, touch sensors, and other components.

U.S. patent application 20240176425 (Wang et al., May 30, 2024, “Method for Controlling Wearable Device and Wearable Device”) discloses detecting an abnormal touch event on a display screen of a wearable device and enabling gesture recognition in response to the abnormal touch event. U.S. patent application 20180123629 (Wetzig, May 3, 2018, “Smart-Ring Methods and Systems”) discloses a computerized smart ring which is embedded with electronics, software, sensors wherein the ring can be electronically connected to another computing system. U.S. patent application 20170071518 (Xavier Da Silveira et al., Mar. 16, 2017, “Apparatus and Method for Optical Tissue Detection”) discloses optical discrimination between body tissue and non-tissue materials.

U.S. patent application 20170303788 (Xavier Da Silveira et al., Oct. 26, 2017, “Wearable Device for Tissue Monitoring With Effective Ambient Light Blocking”) discloses an optical biometric device with a shield to block ambient light. U.S. patent application 20170319131 (Xavier Da Silveira et al., Nov. 9, 2017, “Method and Device for Hydration Monitoring”) discloses using three different wavelengths to measure hydration. U.S. patent application 20190167201 (Xavier Da Silveira et al., Jun. 6, 2019, “Wearable Athletic Monitoring Using Digital Modulation”) discloses a wearable spectroscopic sensor with digital modulation. U.S. patent application 20160338601 (Yang, 2016 Nov. 24, “Optical Fiber Continuous Detecting Blood Sensor and Wearing Apparatus Thereof”) discloses using optical fibers and spectroscopic sensors to measure blood pressure.

U.S. patent application 20190067257 (Yeon et al., Feb. 28, 2019, “Light-Emitting Diode (LED) Device”) discloses a multi-color display includes a plurality of light-emitting cells at least partially defined by a partition layer. U.S. Pat. No. 11,540,599 (Yokoyama et al., Jan. 3, 2023, “Watch Band with Adjustable Fit”) discloses shape-memory tensioning elements which respond to a stimulus in order to adjust the fit of a watch band. U.S. patent application 20240126382 (Yoo, Apr. 18, 2024, “Wearable Device and Method for Controlling Same”) discloses a method of controlling a smart ring by sensing contact from a finger on an outer surface electrode on an outer circumference of the ring. U.S. Pat. No. 10,357,165 (Yoon, Jul. 23, 2019, “Method and Apparatus for Acquiring Bioinformation and Apparatus for Testing Bioinformation”) discloses a biometric sensor analyzing laser speckle patterns.

### SUMMARY OF THE INVENTION

This invention is a wearable device (such as a smart watch or finger ring) with a circular, annular, or polygonal array of sets of light emitters and light receivers. This array is used to measure one or more of a person's biometric parameters. Light emitted from the emitters is reflected from and/or transmitted through the person's body tissue and then received by the receivers. The spectral distribution of the received light is analyzed to measure one or more of the person's biometric parameters such as oxygenation level, heart rate, heart rate variability, blood pressure, hydration level, and/or blood glucose level.

Each set of light emitters can include three or more emitters which emit light of different colors, wavelengths, and/or frequencies. This device can also include one or more compressible and/or elastomeric opaque light barriers which surround the light receivers. These light barriers can prevent light from the emitters from reaching the receivers without first being reflected from or transmitted through the person's body tissue. These light barriers can also prevent ambient light from reaching the light receivers.

### BRIEF INTRODUCTION TO THE FIGURES

FIG. 1 shows a smart watch with a circular and/or annular array of light emitters around a light receiver.

FIG. 2 shows a smart watch with a circular and/or annular array of alternating light emitters and light receivers.

FIG. 3 shows an arcuate band with a circular and/or annular array of light emitters around a light receiver.

FIG. 4 shows an arcuate band with a circular and/or annular array of light receivers around a light emitter.

FIG. 5 shows an arcuate band with a circular and/or annular array of light receivers, a central light emitter, and a light barrier between the light receivers and the light emitter.

FIG. 6 shows an arcuate band with a circular and/or annular array of light receivers, a central light emitter, a light barrier between the light receivers and the light emitter, and a light barrier between the light receivers and ambient light.

### DETAILED DESCRIPTION OF THE FIGURES

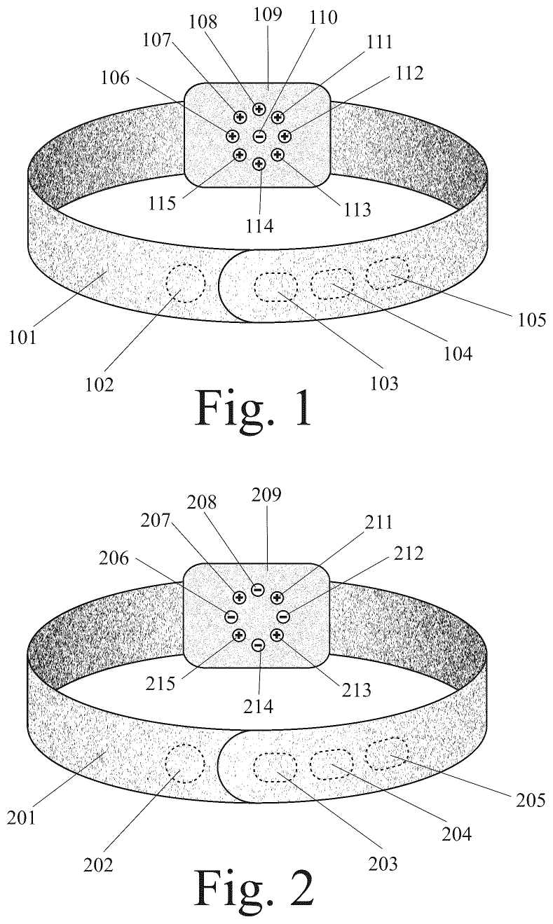

Before discussing the specific embodiments of this invention which are shown in FIGS. 1 through 6, this disclosure provides an introductory section which covers the general concepts, components, and methods which comprise this invention. Where relevant, these concepts, components, and methods can be applied as variations to the examples shown in FIGS. 1 through 6 which are discussed afterwards.

In an example, a wearable device for measuring one or more of a person's biometric parameters can comprise: a housing which is configured to be worn on a person's wrist, arm, or finger; an array of light emitter sets and light receivers on the housing; wherein light emitted from the light emitters is reflected from and/or transmitted through the person's body tissue and received by the light receivers; wherein attributes of the light received by the light receivers are analyzed to measure one or more biometric parameters of the person; wherein there is an alternating pattern of light emitter sets and light receivers in the array; and wherein at least one light emitter in a set emits red light, at least one light emitter in the set emits infrared or near-infrared light, and at least one light emitter in the set emits green light; a data processor; and one or more opaque light barriers surrounding one or more light receivers, wherein the light barriers are compressible, compliant, and/or elastomeric.

In an example, a wearable device for measuring one or more of a person's biometric parameters can comprise: a housing which is configured to be worn on a person's wrist, arm, or finger; and an array of light emitter sets and light receivers on the housing, wherein light emitted from the light emitters is reflected from and/or transmitted through the person's body tissue and received by the light receivers, and wherein attributes of the light received by the light receivers are analyzed to measure one or more biometric parameters of the person.

In an example, the device can be a smart watch or wrist band. In an example, the device can be a finger ring. In an example, the array can be a circular, annular, or circumferential array. In an example, the array can be a polygonal array. In an example, the array can be a hexagonal or octagonal array. In an example, there can be an alternating pattern of light emitter sets and light receivers in the array. In an example, there can be three or more light emitters in each light emitter set. In an example, different light emitters in a set can emit light with different colors, wavelengths, and/or frequencies, respectively. In an example, at least one light emitter in a set can emit red light, at least one light emitter in the set can emit infrared or near-infrared light, and at least one light emitter in the set can emit green light. In an example, the attributes of the light which are analyzed can include spectral distribution of the light. In an example, the device can further comprise a data processor. In an example, the device can further comprise one or more opaque light barriers surrounding one or more light receivers.

In an example, the light barriers can be compressible, compliant, and/or elastomeric. In an example, the light barriers can reduce or eliminate transmission of light from the light emitters to the light receivers which has not been reflected by or transmitted through the person's body tissue. In an example, the light barriers can reduce or eliminate transmission of ambient light to the light emitters. In an example, each light barrier can surround a single light receiver. In an example, a single light barrier can surround all of the light receivers.

In an example, a wearable device for measuring one or more of a person's biometric parameters can comprise: an arcuate band which is configured to span a person's wrist, arm, finger; a housing which is attached to the arcuate band; a plurality of light energy emitters which are held by the housing and configured to emit light energy toward the person's body; a central light energy receiver which is held by the housing and configured to receive light energy from the light energy emitters, wherein the plurality of light energy emitters are around the central light energy receiver in an approximately-circular polygonal array; a data processor which is held by the housing and receives data from the light energy receiver; and a data transmitter which is held by the housing and transmits data from the data processor to a remote device and/or remote location.

In an example, a wearable device (e.g. smart watch, watch band, fitness band, or finger ring) for measuring one or more biometric parameters can have a plurality of light emitters and a plurality of light receivers. Light emitters emit light toward a person's body and light receivers can receive this light after it has been reflected by and/or transmitted through the person's body tissue. Changes in light characteristics (e.g. spectral distribution) caused by interaction of the light with the person's body tissue can be analyzed to measure one or more biometric parameters. A light emitter and a light receiver used in this manner can together comprise a spectroscopic sensor. Having a plurality of light emitters and light receivers can enable more accurate measurement of biometric parameters than having just one light emitter and one light receiver because it allows various light path distances and locations in various emitter-to-receiver light pathways.

In an example, the biometric parameter which is measured can be oxygen level (e.g. blood oxygenation). In an example, the biometric parameter which is measured can be heart rate or heart rate variability. In an example, the biometric parameter which is measured can be a person's blood pressure. In an example, the biometric parameter can be body hydration level. In an example, the biometric parameter which is measured can be blood glucose level. In an example, the biometric parameter which is measured can be lactic acid level. In an example, the biometric parameter which is measured can be blood urea nitrogen level (e.g. BUN level).

In an example, a wearable device for measuring one or more biometric parameters can have a plurality of light emitters which are selected from group consisting of: Light Emitting Diode (LED), Organic Light Emitting Diode (OLED), Laser Diode (LD), Superluminescent Light Emitting Diode (SLED), Resonant Cavity Light Emitting Diode (RCLED), coherent light source, infrared (IR) light emitter, laser, low-power laser, microplasma light emitter, multi-wavelength source, Quasi Monochromatic (QM) light, and Ultra Violet (UV) light emitter. In an example, a light receiver can be a photodetector.

In an example, a light emitter can emit coherent light. In an example, a light emitter can be a laser. In an example, a light emitter can be a Light Emitting Diode (LED). In an example, a light emitter can emit infrared or near-infrared light. In an example, a light emitter can emit polarized light. In an example, a light emitter can emit ultraviolet light. In an example, a light emitter emit red light and/or be a red-light laser. In an example, a light emitter emit green light and/or be a green-light laser. In an example, a light emitter can emit white light and/or be a white-light laser. In an example, a light emitter can emit light with a frequency and/or spectrum which changes over time. In an example, a light emitter can emit a sequence of light pulses at different selected frequencies.

In an example, a wearable device can have a plurality of light emitter sets, wherein each set includes a multiple light emitters. In an example, a wearable device can have a plurality of light emitter sets, wherein each set includes a plurality of light emitters which emit light in a plurality of colors, wavelengths, and/or frequencies, respectively. In an example, a wearable device can have a plurality of light emitter sets, wherein each set includes a three light emitters which emit light in three different colors, wavelengths, and/or frequencies, respectively. In an example, a wearable device can have a plurality of light emitter sets, wherein each set includes at least three light emitters including a red light emitter, an infrared (or near infrared) light emitter, and a green light emitter.

In an example, there can be a constant distance between (proximal or adjacent) light emitters in an array and/or configuration of light emitters. In an example, light emitters can be configured in a polygonal array and/or configuration. In an example, light emitters can be configured in a hexagonal or octagonal array and/or configuration. In an example, light emitters can be configured in a circular, annular, and/or nested array and/or configuration. In an example, light emitters can be configured in a radial or hub-and-spoke array and/or configuration. In an example, light emitters can be positioned on a common circumferential line. In an example, light emitters can be positioned on a common proximal-to-distal line, wherein proximal means closer to a person's elbow and distal means farther from the person's elbow.

In an example, there can be a constant distance between (proximal or adjacent) sets of light emitters in an array and/or configuration of sets of light emitters. In an example, sets of light emitters can be configured in a polygonal array and/or configuration. In an example, sets of light emitters can be configured in a hexagonal or octagonal array and/or configuration. In an example, sets of light emitters can be configured in a circular, annular, and/or nested array and/or configuration. In an example, sets of light emitters can be configured in a radial or hub-and-spoke array and/or configuration. In an example, sets of light emitters can be positioned on a common circumferential line. In an example, sets of light emitters can be positioned on a common proximal-to-distal line, wherein proximal means closer to a person's elbow and distal means farther from the person's elbow.

In an example, there can be a constant distance between (proximal or adjacent) light receivers in an array and/or configuration of light receivers. In an example, light receivers can be configured in a polygonal array and/or configuration. In an example, light receivers can be configured in a hexagonal or octagonal array and/or configuration. In an example, light receivers can be configured in a circular, annular, and/or nested array and/or configuration. In an example, light receivers can be configured in a radial or hub-and-spoke array and/or configuration. In an example, light receivers can be positioned on a common circumferential line. In an example, light receivers can be positioned on a common proximal-to-distal line, wherein proximal means closer to a person's elbow and distal means farther from the person's elbow.

In an example, a wearable device can have an array, matrix, or grid of four or more light emitters. In an example, a wearable device can have an array, matrix, or grid of four or more light emitters, each of which is separated from the nearest other light emitter by a distance within the range of 2 mm to 5 cm. In an example, a wearable device can have an array, matrix, or grid of four or more light emitters, each of which is separated from the nearest other light emitter by a (polar and/or angular) distance within the range of 2 degrees to 60 degrees. In an example, a wearable device can have a circular and/or annular array grid of four or more light emitters, each of which is separated from the nearest other light emitter by a (polar and/or angular) distance within the range of 2 degrees to 60 degrees. In an example, a wearable device can have a circular and/or annular array grid of four or more light emitters, each of which is separated from the nearest other light emitter by a (polar and/or angular) distance within the range of 40 degrees to 60 degrees.

In an example, a wearable device can have an array, matrix, or grid of four or more sets of light emitters. In an example, a wearable device can have an array, matrix, or grid of four or more sets of light emitters, each of which is separated from the nearest other light emitter by a distance within the range of 2 mm to 5 cm. In an example, a wearable device can have an array, matrix, or grid of four or more sets of light emitters, each of which is separated from the nearest other light emitter by a (polar and/or angular) distance within the range of 2 degrees to 60 degrees. In an example, a wearable device can have a circular and/or annular array grid of four or more sets of light emitters, each of which is separated from the nearest other light emitter by a (polar and/or angular) distance within the range of 2 degrees to 60 degrees. In an example, a wearable device can have a circular and/or annular array grid of four or more sets of light emitters, each of which is separated from the nearest other light emitter by a (polar and/or angular) distance within the range of 40 degrees to 60 degrees.

In an example, a wearable device can have an array, matrix, or grid of four or more light receivers. In an example, a wearable device can have an array, matrix, or grid of four or more light receivers, each of which is separated from the nearest other light emitter by a distance within the range of 2 mm to 5 cm. In an example, a wearable device can have an array, matrix, or grid of four or more light receivers, each of which is separated from the nearest other light emitter by a (polar and/or angular) distance within the range of 2 degrees to 60 degrees. In an example, a wearable device can have a circular and/or annular array grid of four or more light receivers, each of which is separated from the nearest other light emitter by a (polar and/or angular) distance within the range of 2 degrees to 60 degrees. In an example, a wearable device can have a circular and/or annular array grid of four or more light receivers, each of which is separated from the nearest other light emitter by a (polar and/or angular) distance within the range of 40 degrees to 60 degrees.

In an example, a set of light emitters in an array of light emitters can comprise two or more light emitters. In an example, a set of light emitters in a circumferential array of light emitters can be two or more laterally-proximal light emitters which share the same polar coordinates. In an example, a subset of light emitters in a circumferential array of light emitters can be two or more circumferentially-proximal light emitters with different polar coordinates. In an example, a set of light receivers in an array of light receivers can comprise two or more light receivers. In an example, a set of light receivers in a circumferential array of light receivers can be two or more laterally-proximal light receivers which share the same polar coordinates. In an example, a subset of light receivers in a circumferential array of light receivers can be two or more circumferentially-proximal light receivers with different polar coordinates.

In an example, light emitters in a circumferential array can be equally spaced and/or distributed around (a portion of) the circumference of an arcuate band. In an example, proximal pairs of light emitters in a circumferential array on an arcuate band can be separated by the same number of polar degrees and/or the same distance. In an example, a circumferential array of light emitters can comprise 24, 12, 8, 6, or 4 emitters. In an example, proximal pairs of light emitters in a circumferential array can have polar coordinates which differ by 15, 30, 40, 60, or 90 degrees.

In an example, sets of light emitters in a circumferential array can be equally spaced and/or distributed around (a portion of) the circumference of an arcuate band. In an example, proximal pairs of sets of light emitters in a circumferential array on an arcuate band can be separated by the same number of polar degrees and/or the same distance. In an example, a circumferential array of sets of light emitters can comprise 24, 12, 8, 6, or 4 emitters. In an example, proximal pairs of sets of light emitters in a circumferential array can have polar coordinates which differ by 15, 30, 40, 60, or 90 degrees.

In an example, light receivers in a circumferential array can be equally spaced and/or distributed around (a portion of) the circumference of an arcuate band. In an example, proximal pairs of light receivers in a circumferential array on an arcuate band can be separated by the same number of polar degrees and/or the same distance. In an example, a circumferential array of light receivers can comprise 24, 12, 8, 6, or 4 receivers. In an example, proximal pairs of light receivers in a circumferential array can have polar coordinates which differ by 15, 30, 40, 60, or 90 degrees.

In an example, a wearable device can have a two-dimensional array of spectroscopic sensors (e.g. paired light emitters or sets of light emitters and light receivers). In an example, spectroscopic sensors in a two-dimensional array can differ in location circumferentially or annularly (e.g. be at different locations around the circumference of a wearable device) and laterally (e.g. be at different locations along axes which are perpendicular to the circumference of the device). In an example, a circumferential or annular array of light receivers can be evenly spaced or distributed, with the same pair-wise distance or number of degrees between adjacent light receivers.

In an example, a wearable device can comprise a plurality of sets of light emitters and light receivers, wherein each set forms a vertex of a hexagon. In an example, a wearable device can comprise a plurality of sets of light emitters and light receivers, wherein each set forms a vertex of an octagon. In an example, a wearable device can comprise an alternating sequence of light emitters and light receivers, wherein each emitter or receiver is located at a vertex of a polygon (e.g. hexagon or octagon). In an example, a wearable device can comprise an alternating sequence of sets light emitters and light receivers, wherein each emitter or receiver is located at a vertex of a polygon (e.g. hexagon or octagon). In an example, a substantially circular and/or annular array can be formed from a plurality of (sets of) light emitters and light receivers, wherein each forms a point on the circle.

In an example, a wearable device can have a circumferential, annular, and/or circular array of light emitters and receivers along a single circumferential axis (e.g. circumferential line). In an example, a wearable device can have a first circumferential or annular array of light emitters and light receivers around a first circumferential axis and a second circumferential or annular array of light receivers around a second circumferential axis, wherein the first and second circumferential axes are parallel to each other. In an example, a wearable device can have two or more circumferential or annular arrays of light emitters and light receivers around circumferential axes which are parallel to each other.

In an example, a wearable device can comprise an array of light emitters and light receivers on a housing (e.g. smart watch housing) which is held on a person's body by an arcuate band or strap (e.g. watch strap) which, in turn, is fastened onto the person's body. In an example, a wearable device can comprise an array of light emitters and light receivers on an arcuate band or strap (e.g. watch strap or fitness band) which is fastened onto a person's body. In an example, a wearable device can comprise an array of light emitters and light receivers on both a housing (e.g. watch housing) and an arcuate band or strap (e.g. watch strap or fitness band) which holds the housing onto a person's body.

In an example, a wearable device can comprise an annular and/or circular array of light emitters and light receivers on a housing (e.g. watch housing) which is held on a person's body by an arcuate band or strap (e.g. watch strap) which, in turn, is fastened onto the person's body. In an example, a wearable device can comprise an annular, circular, and/or circumferential array of light emitters and light receivers on an arcuate band or strap (e.g. watch strap or fitness band) which is fastened onto a person's body. In an example, a wearable device can comprise an annular, circular, and/or circumferential array of light emitters and light receivers on both a housing (e.g. watch housing) and on an arcuate band or strap (e.g. watch strap or fitness band) which holds the housing onto a person's body.

In an example, a wearable device can comprise an annular and/or circular alternating (e.g. emitter then receiver) array of light emitters and light receivers on a housing (e.g. watch housing) which is held on a person's body by an arcuate band or strap (e.g. watch strap) which, in turn, is fastened onto the person's body. In an example, a wearable device can comprise an annular, circular, and/or circumferential alternating (e.g. emitter then receiver) array of light emitters and light receivers on an arcuate band or strap (e.g. watch strap or fitness band) which is fastened onto a person's body. In an example, a wearable device can comprise an annular, circular, and/or circumferential alternating (e.g. emitter then receiver) array of light emitters and light receivers on both a housing (e.g. watch housing) and on an arcuate band or strap (e.g. watch strap or fitness band) which holds the housing onto a person's body.

In an example, different combinations of light emitters in a circumferential or annular array can be activated at different times in order to measure combined effects of light reflected from, or having passed through, the body along different light pathways. In an example, activating light emitters in different locations around the circumferential or annular array can enable light beam triangulation to measure biometric parameter levels in specific (cross-sectional) areas of a person's finger, wrist, and/or arm.

In an example, a circumferential, annular, and/or circular array of light emitters and light receivers can enable analysis of variation in biometric parameter levels by tissue depth and location. This provides richer information on biometric parameter levels within body tissue than is possible with a single light emitter and receiver pair. Incorporating multiple light emitters around the circumference of a wearable device provides data on biometric parameter levels at different locations and tissue depths. It can also help to control for shifts of the device on (or around) a person's finger, wrist, and/or arm.

In an example, as different light emitters are activated in an array on a person's wrist, finger, and/or arm, cross-sectional (and/or volumetric) scan of biometric parameter levels can be possible. In an example, different combinations of light emitters in a circumferential array can be activated in order to measure combined effects of light reflected from, or having passed through, the body along different pathways. In an example, activating light emitters in different locations around a circumferential array can enable light beam triangulation to measure biometric parameter levels in specific (cross-sectional) areas of a person's wrist, finger, and/or arm.

In an example, a wearable device can comprise a first set of light emitters of a first type and a second circumferential array of light emitters of a second type, wherein the first and second types of light emitters differ from each other by one or more characteristics selected from the group consisting of: light beam color and/or spectral frequency; light beam intensity and/or power; size and/or shape; angle of light beam projection; light coherence; and light polarity.

In an example, a wearable device can comprise an array, grid, and/or matrix of light emitters which differ in one or more parameters selected from the group consisting of: location and/or distance from a light receiver; distance to body surface; light beam frequency, color, and/or spectrum; light beam coherence, polarity, and/or phase; light beam power and/or intensity; light beam projection and/or body incidence angle; light beam duration; light beam size; and light beam focal distance. In an example, a wearable device can comprise an array, grid, and/or matrix of light receivers which differ in: location and/or distance from a light emitter; and/or distance to body surface.

In an example, a first light emitter can emit light with a first light power and/or intensity and a second light emitter can emit light with a second light power and/or intensity. In an example, a first light emitter can emit light with a first light projection and/or body incidence angle and a second light emitter can emit light with a second light projection and/or body incidence angle. In an example, a first light emitter can emit light with a first light coherence, polarization, and/or phase and a second light emitter can emit light with a second light coherence, polarization, and/or phase.

In an example, a wearable device can include light emitters which emit light of different colors, at different wavelengths, at different frequencies, and/or with different light spectral distributions. In an example, different emitters can emit light with different wavelengths or wavelength ranges. In an example, different emitters can emit light with different wavelengths or wavelength ranges to detect different biometric parameters or physiological conditions. In an example, different emitters can emit light with different wavelengths or wavelength ranges based on when a person is engaged in different types of activities. In an example, different emitters can emit light with different wavelengths or wavelength ranges based in response to different environmental parameters or conditions.

In an example, a first light emitter can emit light with a first frequency and/or spectrum and a second light emitter can emit light with a second frequency and/or spectrum, wherein the second frequency and/or spectrum is different than the first frequency and/or spectrum. In an example, the frequency and/or spectrum of light emitted by a light emitter can be changed over time. In an example, a wearable device can comprise a spectroscopic sensor (e.g. spectrometer) which emits light at different frequencies at different times. In an example, a wearable device can comprise a spectroscopic sensor which emits a sequence of light at different frequencies. In an example, a light emitter can emit light with scanning variation in frequencies and/or wavelength.

In an example, a light emitter of a spectroscopic sensor can emit light in a sweeping series of frequencies. In an example, a light emitter of a spectroscopic sensor can emit light in a sequentially-varying range of frequencies. In an example, a light emitter of a spectroscopic sensor can emit light with a frequency which changes over time. In an example, a light emitter of a spectroscopic sensor can emit light in a sweeping series of wavelengths. In an example, a light emitter of a spectroscopic sensor can emit light in a sequentially-varying range of wavelengths. In an example, a light emitter of a spectroscopic sensor can emit light with a wavelength which changes over time.

In an example, a spectroscopic sensor (e.g. spectrometer) can comprise a plurality of light emitters which emit light in different wavelength ranges. In an example, a spectroscopic sensor can comprise a plurality of light emitters which emit light at different frequencies. In an example, a wearable device can comprise a plurality of spectroscopic sensors which sequentially emit light at different frequencies. In an example, a wearable device can comprise a plurality of spectroscopic sensors which simultaneously emit light at different frequencies. In an example, the operation of a spectroscopic sensor can include frequency-based modulation.

In an example, there can be differences in the wavelengths, colors, and/or spectra of light emitted by light emitters at different circumferential locations in an array. In an example, a first light emitter in a wearable ring of biometric sensors can emit light with a first wavelength, color, and/or spectrum and a second light emitter in the wearable ring of biometric sensors can emit light with a second wavelength, color, and/or spectrum.

In an example, there can be alternation between two light wavelengths and/or colors in an arcuate array of light emitters around (at least half of) the circumference of a person's wrist, finger, or arm. In an example, there can be a repeating sequence of two different light wavelengths and/or colors in an arcuate array of light emitters around (at least half of) the circumference of a person's wrist, finger, or arm. In an example, there can be a repeating sequence of three different light wavelengths and/or colors in an arcuate array of light emitters around (at least half of) the circumference of a person's wrist, finger, or arm.

In an example, a wearable device can comprise at least two light emitters which emit light of different colors, respectively. In an example, a first light emitter in a wearable device can emit red light and a second light emitter in the ring can emit infrared light. In an example, a first light emitter in a wearable device can be a red LED and a second light emitter in the ring can be an infrared LED. In an example, a first light emitter in a wearable device can emit red light and a second light emitter in the ring can emit green light. In an example, a first light emitter in a wearable device can be a red LED and a second light emitter in the ring can be a green LED. In an example, a first light emitter in a wearable device can emit infrared light and a second light emitter in the ring can emit green light. In an example, one light emitter in a wearable device can be an infrared LED and a second light emitter in the ring can be a green LED.

In an example, a first light emitter which emits light in a first color can emit light with a first intensity or power level and a second light emitter which emits light in a second color can emit light with a second intensity or power level. In an example, a first light emitter which emits light in a first color can emit light at a first time and a second light emitter which emits light in a second color can emit light at a second time. In an example, a first light emitter which emits light in a first color can emit light with a first oscillation frequency and a second light emitter which emits light in a second color can emit light with a second oscillation frequency. In an example, a first light emitter which emits light in a first color can emit light along a first angle and/or vector and a second light emitter which emits light in a second color can emit light along a second angle and/or vector.

In an example, a wearable device can collect data which is used to measure a person's blood and/or tissue oxygenation. In an example, a wearable device with two light emitters which emit light with two different colors and/or wavelengths can be used to measure a person's blood and/or tissue oxygenation level. In an example, a wearable device with two light emitters which emit light with two different colors and/or wavelengths can be used to measure other biometric parameters such as heart rate, heart rate variability, blood pressure, body hydration, and/or blood glucose level.

In an example, a wearable device can comprise a plurality of sets of light emitters and light receivers which collectively span (at least half) of the circumference of a person's wrist, finger, or arm, wherein light from one or more light emitters in a set is received by one or more light receivers in that set. In an example, different light emitters in a set can emit light at different frequencies. In an example, each set can have a first light emitter which emits light with a first frequency and a second light emitter which emits light with a second frequency. In an example, a wearable device can comprise between 4 and 10 sets of light emitters and light receivers. In an example, a wearable device can comprise between 6 and 24 sets of light emitters and light receivers.

In an example, a wearable device can comprise at least two light emitters which emit light at different wavelengths. In an example, a wearable device can comprise at least three light emitters which emit light at different wavelengths. In an example, a wearable device can comprise: a first light emitter which emits light at a wavelength in the range of 400 nm to 600 nm; and a second light emitter which emits light at a wavelength in the range of 680 nm to 880 nm. In an example, a wearable ring can comprise: a first light emitter which emits light at a wavelength in the range of 400 nm to 600 nm; and a second light emitter which emits light at a wavelength in the range of 780 nm to 900 nm.

In an example, a first light emitter in a device can emit light at a wavelength in the range of 600 nm to 690 nm, a second light emitter in the device can emit light at a wavelength in the range of 710 nm to 950 nm, and a third light emitter in the device can emit light at a wavelength in the range of 1000 nm to 1500 nm. In an example, a wearable device can comprise at least three light emitters which emit light of different colors. In an example, one light emitter in a device can emit red light, a second light emitter in a device can emit infrared light, and a third light emitter in a device can emit green light. In an example, one light emitter in a device can be a red LED, a second light emitter in a device can be an infrared LED, and a third light emitter in a device can be a green LED.

In an example, one or more light emitters in a device can emit red light. In an example, one or more light emitters in a device can be a red LED. In an example, one or more light emitters in a device can emit infrared light. In an example, one or more light emitters in a device can be an infrared LED. In an example, one or more light emitters in a device can emit green light. In an example, one or more light emitters in a device can be a green LED.

In an example, a first light emitter can emit light with a first light frequency, color, and/or spectrum and a second light emitter can emit light with a second light frequency, color, and/or spectrum. In an example, light from the first light emitter can reflect primarily from a first depth, breadth, location, and/or type of body tissue and light from the second light emitter can reflect primarily from a second depth, breadth, location, and/or type of body tissue. In an example, first and second light emitters can emit light simultaneously. In an example, first and second light emitters can emit light in a selected chronological sequence and/or timing pattern.

In an example, a wearable device can comprise a plurality of light emitters which emit light toward a person's body at a plurality of different angles, respectively. In an example, different light emitters can emit light toward a person's body at different angles. In an example, a wearable device can comprise a plurality of light emitters which emit light toward a person's body at a plurality of different angles with respect to a wearable device housing, strap, or band. In an example, different light emitters can emit light toward a person's body at different angles with respect to a wearable device housing, strap, or band. In an example, a wearable device can comprise a plurality of light emitters which emit light toward a person's body at a plurality of different angles with respect to a proximal surface of a person's body. In an example, different light emitters can emit light toward a person's body at different angles with respect to a proximal surface of a person's body.

In an example, the first light emitter can emit light at a first angle with respect to the surface of a person's body and the second light emitter can emit light at a second angle with respect to the surface of a person's body. In an example, different emitters in this array can emit light at different angles based on different biometric parameters or physiological conditions. In an example, different emitters in this array can emit light at different angles based different types of activities in which a person is engaging. In an example, different emitters in this array can emit light at different angles based on different environmental parameters or conditions.

In an example, a light emitter can emit light along a first vector and a light receiver can receive light along a second vector. In an example, the second vector can be substantially reversed from and parallel to the first vector. In an example, a beam of light can: be emitted by the light emitter along a first vector; pass through the first (transmissive) side of an angled one-way mirror; hit body tissue; reflect back from the body tissue; reflect off the second (reflective) side of the angled one-way mirror; reflect off a second mirror; and enter the light receiver along a second vector which is reversed from and parallel to the first vector.

In an example, different light emitters in a wearable device can emit beams of light which reach the surface of a person's body at different angles and/or along different vectors with respect to the surface of the person's body. In an example, a first light emitter can emit beams of light which reach the surface of a person's body at a first angle and/or vector with respect to the surface of the person's body and a second light emitter can emit beams of light which reach the surface of the person's body at a second angle and/or vector with respect to the surface of the person's body. In an example, the second angle can differ from the first angle by a difference in the range of 10 to 40 degrees. In an example, the second angle can differ from the first angle by a difference in the range of 30 to 60 degrees. In an example, a first angle can be 90 degrees and a second angle can be in the range of 70 to 80 degrees. In an example, a first angle can be in the range of 90 to 110 degrees and a second angle can be in the range of 70 to 90 degrees.

In an example, a wearable device can comprise a circular or elliptical array, grid, and/or matrix of alternating light emitters and receivers. In an example, a wearable device can comprise a checkerboard array, grid, and/or matrix of alternating light emitters and receivers. In an example, a wearable device can comprise a three-dimensional stacked array, grid, and/or matrix of alternating light emitters and receivers. In an example, a wearable device can comprise a sunburst and/or radial-spoke array, grid, and/or matrix of alternating light emitters and receivers. In an example, a wearable device can comprise a sinusoidal array, grid, and/or matrix of alternating light emitters and receivers.

In an example, a wearable device can comprise an alternating sequence of light emitters and light receivers along a circumference of a smart watch housing, smart watch band, or finger ring. In an example, a wearable device can comprise at least two sensor pairs along a circumference of a finger ring, wrist band, or watch, wherein each sensor pair comprises a light emitter and a light receiver. In an example, data from a sequentially-activated circumferential or annular array of light emitters and light receivers can be used to create a two-dimensional image showing variation in biometric parameter levels within a virtual cross-section of a person's finger, wrist, and/or arm.

In an example, a wearable device can comprise: a wearable device worn around a body member; two or more nested arcuate arrays (e.g. rings) of light emitters and light receivers around the device, wherein each arcuate array comprises an alternating sequence of light emitters and light receivers around the device, wherein the light receivers receive light beams from the light emitters after the light beams have been reflected by (or passed through) the body member, and wherein changes in the spectra of the light beams are analyzed to measure biometric parameter level.

In an example, a wearable device can comprise: a wearable device worn around a body member; two or more concentric arcuate arrays (e.g. rings) of light emitters and light receivers around the device, wherein each arcuate array comprises an alternating sequence of light emitters and light receivers around the device, wherein the light receivers receive light beams from the light emitters after the light beams have been reflected by (or passed through) the body member, and wherein changes in the spectra of the light beams are analyzed to measure biometric parameter level.

In an example, there can be an alternating sequence of light emitters and light receivers around a circumferential line around the circumference of a device and/or around (at least half of) the circumference of a person's wrist, finger, or arm. In an example, there can be a repeating sequence of light emitters and light receivers around a circumferential line around (at least half of) the circumference of a device and/or around the circumference of a person's wrist, finger, or arm. In an example, there can be proximal (e.g. adjacent or nearby) pairs of light emitters and light receivers. In an example, an array can be a circumferential array of pairs of light emitters and light receivers. In an example, a paired light emitter and light receiver can both be located on a line which is orthogonal to a circumferential line of the device.

In an example, a wearable device can comprise a linear array, grid, and/or matrix of light emitters. In an example, a wearable device can comprise a rectangular array, grid, and/or matrix of light emitters. In an example, a wearable device can comprise a circular or elliptical array, grid, and/or matrix of light emitters. In an example, a wearable device can comprise a checkerboard array, grid, and/or matrix of light emitters. In an example, a wearable device can comprise a three-dimensional stacked array, grid, and/or matrix of light emitters. In an example, a wearable device can comprise a sunburst and/or radial-spoke array, grid, and/or matrix of light emitters. In an example, a wearable device can comprise a sinusoidal array, grid, and/or matrix of light emitters.

In an example, a wearable device can comprise a linear array, grid, and/or matrix of light receivers. In an example, a wearable device can comprise a rectangular array, grid, and/or matrix of light receivers. In an example, a wearable device can comprise a circular or elliptical array, grid, and/or matrix of light receivers. In an example, a wearable device can comprise a checkerboard array, grid, and/or matrix of light receivers. In an example, a wearable device can comprise a three-dimensional stacked array, grid, and/or matrix of light receivers. In an example, a wearable device can comprise a sunburst and/or radial-spoke array, grid, and/or matrix of light receivers. In an example, a wearable device can comprise a sinusoidal array, grid, and/or matrix of light receivers.

In an example, a plurality of light emitters can be configured in a polygonal array including a light receiver. In an example, a plurality of light emitters can be configured in a polygonal array around a light receiver. In an example, a plurality of light emitters can be configured in a circular or other arcuate array including a light receiver. In an example, a plurality of light emitters can be configured in a circular or other arcuate array around a light receiver. In an example, a plurality of light emitters can emit light in a circular sequence around a central light receiver.

In an example, a plurality of light receivers can be configured in a linear array including a light emitter. In an example, a plurality of light receivers can be configured in a polygonal array in proximity to a light emitter. In an example, a plurality of light receivers can be configured in a polygonal array including a light emitter. In an example, a plurality of light receivers can be configured in a polygonal array around a light emitter. In an example, a plurality of light receivers can be configured in a circular or other arcuate array including a light emitter. In an example, a plurality of light receivers can be configured in a circular or other arcuate array around a light emitter.

In an example, an array of light emitters and light receivers can have a square or rectangular shape. In an example, an array of light emitters and light receivers can have a hexagonal shape. In an example, an array of light emitters and light receivers can have a circular shape. In an example, an array of light emitters and light receivers can have a sunburst (e.g. radial spoke) shape. In an example, an array of light emitters and light receivers can have a cylindrical and/or ring shape. In an example, an array of light emitters and light receivers can have a conic section shape. In an example, an array of light emitters and light receivers can have a saddle shape. In an example, an array of light emitters and light receivers can have a helical shape.

In an example, a first light emitter of a device can emit light at a first time and a second light emitter of the device can emit light at a second time. In an example, a first subset of light emitters can emit light at a first time and a second subset of light emitters can emit light at a second time. In an example, a wearable device can have light emitters which emit light in a non-simultaneous (e.g. sequential) manner.

In an example, a wearable device can have a circumferential, annular, and/or circular array of sequentially-activated light emitters. In an example, light emitters in a circumferential, annular, and/or circular array can be activated to shine, one light at a time. In an example, light emitters in a circumferential array can be activated in a sequence like moving lights on a theater marquee. In an example, light emitters in a circumferential array can be activated in a clockwise (or counter-clockwise) sequence. In an example, data from a sequentially-activated circumferential array of light emitters and light receivers can be used to create a two-dimensional image showing variation in biometric parameter levels within a virtual cross-section of a person's finger, wrist, and/or arm.

In an example, a first light at a first polar coordinate can be activated to emit light at a first time and second light at a second polar coordinate can be activated to emit light at a second time. In an example, a first light at a first polar coordinate can be activated to emit light at a first time and second light at a second polar coordinate can be activated to emit light at a second time, wherein the second light is the light in the array which is clockwise (or counter-clockwise) most proximal to the first light. In an example, a clockwise (or counter-clockwise) sequence of light can be activated to cause a series of light emissions which encircle the circumference of the arcuate band and/or the finger, wrist, or arm.

In an example, the most accurate locations for measuring a biometric parameter can be identified depending on the current configuration of a wearable device on the person's finger, wrist, and/or arm. This can help to control for shifting and/or rotation of a wearable device with respect to a person's finger, wrist, and/or arm. In an example, if a particular area of a person's finger, wrist, or arm is best for measuring analyte concentration, but the band shifts over time, then pattern recognition can be done to identify which subset of light emitters are over that particular area at a given time and sequential light activation can be concentrated on that area.

In an example, activating different subsets of light emitters at different times can enable a light receiver to separately analyze light beams which have been reflected from, or transmitted through, body tissue along different pathways. In an example, there can be pauses between the times when different subsets of light emitters are activated in a circumferential array. In an example, there can be overlap in the times that different subsets of light emitters are activated in a circumferential array. In an example, the lengths of time wherein light emitters are activated can be changed and/or adjusted to refine measurement of biometric parameter levels.

In an example, the color and/or spectrum of light from light emitters can be changed and/or adjusted to refine measurement of biometric parameter levels. In an example, the duration of activation of different subsets of light emitters in a circumferential array can be adjusted based on analysis of light spectra. In an example, the speed with which light emitters in a circle around a finger, wrist, or arm can be sequentially activated in a clockwise (or counter-clockwise) manner can be increased or decreased based on analysis of light spectra.

In an example, a first subset of one or more light emitters selected from an array of light emitters can have a first average polar coordinate and emit light at a first point in time and a second subset of one or more light emitters selected from an array of light emitters can have a second average polar coordinate and emit light at a second point in time. The second average coordinate can differ from the first average coordinate by at least 5 degrees. The light receivers can be configured to receive light from the first subset of light emitters at a first point in time after light from this subset has interacted with the person's body tissue and the light receivers can be configured to receive light from the second subset of light emitters at a second point in time after light from this subset has interacted with the person's body tissue.

In an example, the first light emitter can emit light during a first environmental condition and the second light emitter can emit light during a second environmental condition. In an example, the first light emitter can emit light when the person is engaged in a first type of physical activity and the second light emitter can emit light when the person is engaged in a second type of physical activity. In an example, different emitters in an array can emit light based on biometric parameters or physiological conditions. In an example, different emitters in an array can emit light when a person is engaged in different types of activities. In an example, different emitters in an array can emit light based on different environmental parameters or conditions.

In an example, a wearable device can comprise: an annular array which alternates between light emitters and light receivers around the device, wherein light emitters in the array are activated to emit light at different times in a clockwise (or counter-clockwise) sequence, wherein light receivers receive light beams from light emitters after the light beams have been reflected by (or passed through) the body member, and wherein a biometric parameter level is measured by changes in the spectra of the light beams.

In an example, a wearable device can comprise a plurality of sets of light emitters and light receivers, wherein different sets are activated at different times. In an example, a wearable device can comprise a plurality of sets of light emitters and light receivers around the circumference of a person's wrist, finger, or arm, wherein different sets are activated sequentially in a clockwise (or counter-clockwise) manner around the circumference of the person's wrist, finger, or arm. In an example, a plurality of light emitters in a set can be distributed around a light receiver in a circular or polygonal pattern. In an example, a plurality of light receivers in a set can be distributed around a light emitter in a circular or polygonal pattern.

In an example, a wearable device can have a light emitter which emits light with a first light wavelength (or wavelength range or spectral distribution) during a first time period and emits light with a second light wavelength (or wavelength range or spectral distribution) during a second time period. In an example, a light emitter can emit light with a first light wavelength (or wavelength range or spectral distribution) during a first time period and can emit light with a second light wavelength (or wavelength range or spectral distribution) during a second time period in order to measure different biometric parameters or physiological conditions.

In an example, a light emitter can emit light with a first light wavelength (or wavelength range or spectral distribution) during a first time period and can emit light with a second light wavelength (or wavelength range or spectral distribution) during a second time period in response to changing environmental conditions. In an example, a light emitter can emit light with a first light wavelength (or wavelength range or spectral distribution) during a first time period and can emit light with a second light wavelength (or wavelength range or spectral distribution) during a second time period in response to changing biometric parameter results. In an example, a light emitter can emit light with a first light wavelength (or wavelength range or spectral distribution) during a first time period and can emit light with a second light wavelength (or wavelength range or spectral distribution) during a second time period in response to changing physiological conditions.

In an example, there can be changes in the wavelength, color, and/or spectrum of light emitted by the same light emitter over time. In an example, a light emitter in a wearable ring of biometric sensors can emit light with a first wavelength, color, and/or spectrum at a first time and emit light with a second wavelength, color, and/or spectrum at a second time. In an example, the wavelength, color, and/or spectrum of light emitted by a light emitter can be automatically oscillated and/or iteratively-varied by a device in order to scan body tissue at different depths and/or locations. In an example, the wavelength, color, and/or spectrum of light emitted by a light emitter can be automatically oscillated and/or iteratively-varied by the device in order to scan body tissue at different spectral wavelengths. In an example, the wavelength, color, and/or spectrum of light emitted by a light emitter can be automatically oscillated and/or iteratively-varied by a device.

In an example, the depth, breadth, location, and/or type of body tissue or fluid from which light from a light emitter is reflected can be changed by adjusting the frequency, color, and/or spectrum of light emitted from the light emitter. In an example, the frequency, color, and/or spectrum of light emitted from the light emitter can be adjusted in order to more accurately measure body oxygenation, heart rate, heart rate variability, body hydration level, and/or blood glucose level. In an example, the frequency, color, and/or spectrum of light emitted from the light emitter can be adjusted automatically (in an iterative manner) by a device in order to more accurately measure body oxygenation, heart rate, heart rate variability, body hydration level, and/or blood glucose level for a specific person, for a specific type of activity, or for a specific configuration of the device relative to the person's body surface.

In an example, the frequency, color, and/or spectrum of light emitted from the light emitter can be adjusted automatically to maintain accurate measurement of body oxygenation, heart rate, heart rate variability, body hydration level, and/or blood glucose level even if the device shifts and/or moves relative to the person's body surface. In an example, a wearable device can automatically vary the frequency, color, and/or spectrum of light from a light emitter to scan through a range of tissue depths, locations, and/or types in order to obtain more accurate measurement of body oxygenation, heart rate, heart rate variability, body hydration level, and/or blood glucose level. In an example, a wearable device can further comprise one or more optical filters or lenses which change the frequency, color, and/or spectrum of light emitted by a light emitter.

In an example, the depth, breadth, location, and/or type of body tissue or fluid from which light from a light emitter is reflected can be changed by adjusting the power and/or intensity of light emitted from the light emitter. In an example, the power and/or intensity of light emitted from the light emitter can be adjusted in order to more accurately measure body oxygenation, heart rate, heart rate variability, body hydration level, and/or blood glucose level. In an example, the power and/or intensity of light emitted from the light emitter can be adjusted automatically (in an iterative manner) by a device in order to more accurately measure body oxygenation, heart rate, heart rate variability, body hydration level, and/or blood glucose level for a specific person, for a specific type of activity, or for a specific configuration of the device relative to the person's body surface.

In an example, the power and/or intensity of light emitted from the light emitter can be adjusted automatically to maintain accurate measurement of body oxygenation, heart rate, heart rate variability, body hydration level, and/or blood glucose level even if the device shifts and/or moves relative to the person's body surface. In an example, a wearable device can automatically vary the power and/or intensity of light from a light emitter to scan through a range of tissue depths, locations, and/or types in order to obtain more accurate measurement of body oxygenation, heart rate, heart rate variability, body hydration level, and/or blood glucose level.

In an example, the depth, breadth, location, and/or type of body tissue or fluid from which light from a light emitter is reflected can be changed by adjusting the coherence, polarization, and/or phase of light emitted from the light emitter. In an example, the coherence, polarization, and/or phase of light emitted from the light emitter can be adjusted in order to more accurately measure body oxygenation, heart rate, heart rate variability, body hydration level, and/or blood glucose level. In an example, the coherence, polarization, and/or phase of light emitted from the light emitter can be adjusted automatically (in an iterative manner) by a device in order to more accurately measure body oxygenation, heart rate, heart rate variability, body hydration level, and/or blood glucose level for a specific person, for a specific type of activity, or for a specific configuration of the device relative to the person's body surface.

In an example, the coherence, polarization, and/or phase of light emitted from the light emitter can be adjusted automatically to maintain accurate measurement of body oxygenation, heart rate, heart rate variability, body hydration level, and/or blood glucose level even if the device shifts and/or moves relative to the person's body surface. In an example, a wearable device can automatically vary the coherence, polarization, and/or phase of light from a light emitter to scan through a range of tissue depths, locations, and/or types in order to obtain more accurate measurement of body oxygenation, heart rate, heart rate variability, body hydration level, and/or blood glucose level. In an example, a wearable device can further comprise one or more optical filters or lenses which change the coherence, polarization, and/or phase of light emitted by a light emitter.

In an example, the frequency, color, and/or spectrum of a beam of light emitted from a light emitter can be changed over time to create a chronological sequence of beams of light with different frequencies, colors, and/or spectrums. In an example, the angle of a beam of light emitted from a light emitter can be changed over time to create a chronological sequence of beams of light with different projection and/or body incidence angles. In an example, the power or intensity of a beam of light emitted from a light emitter can be changed over time to create a chronological sequence of beams of light with different power or intensity levels. Such sequences can help to more accurately measure body oxygenation, heart rate, heart rate variability, body hydration level, and/or blood glucose level.

In an example, the frequency, color, and/or spectrum of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure body oxygenation, heart rate, heart rate variability, body hydration level, and/or blood glucose level.

In an example, the projection angle of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure body oxygenation, heart rate, heart rate variability, body hydration level, and/or blood glucose level.

In an example, the power and/or intensity of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure body oxygenation, heart rate, heart rate variability, body hydration level, and/or blood glucose level.

In an example, the frequency, color, and/or spectrum of light emitted from a light emitter can be adjusted automatically (in an iterative manner) by a device in order to more accurately measure biometric parameter level for a specific person, for a specific type of activity, or for a specific configuration of the device relative to the person's body surface.

In an example, the frequency, color, and/or spectrum of light emitted from the light emitter can be adjusted automatically to maintain accurate measurement of biometric parameter level even if the device shifts and/or moves relative to the person's body surface. In an example, a wearable device can automatically vary the frequency, color, and/or spectrum of light from a light emitter to scan through a range of tissue depths, locations, and/or types in order to obtain more accurate measurement of biometric parameter level.

In an example, the power and/or intensity of light emitted from the light emitter can be adjusted in order to more accurately measure biometric parameter level. In an example, the power and/or intensity of light emitted from the light emitter can be adjusted automatically (in an iterative manner) by a device in order to more accurately measure biometric parameter level for a specific person, for a specific type of activity, or for a specific configuration of the device relative to the person's body surface.

In an example, the coherence, polarization, and/or phase of light emitted from the light emitter can be adjusted in order to more accurately measure biometric parameter level. In an example, the coherence, polarization, and/or phase of light emitted from the light emitter can be adjusted automatically (in an iterative manner) by a device in order to more accurately measure biometric parameter level for a specific person, for a specific type of activity, or for a specific configuration of the device relative to the person's body surface.

In an example, the coherence, polarization, and/or phase of light emitted from the light emitter can be adjusted automatically to maintain accurate measurement of biometric parameter level even if the device shifts and/or moves relative to the person's body surface. In an example, a wearable device can automatically vary the coherence, polarization, and/or phase of light from a light emitter to scan through a range of tissue depths, locations, and/or types in order to obtain more accurate measurement of biometric parameter level.

In an example, the frequency, color, and/or spectrum of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure biometric parameter level.

In an example, the projection angle of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure biometric parameter level. In an example, the power and/or intensity of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure biometric parameter level.

In an example, the angle at which light is emitted from a light emitter leaves a device and/or the angle at which the light intersects the surface of a person's body can be automatically changed over time. In an example, the angle at which light is emitted from a light emitter leaves a device and/or the angle at which the light intersects the surface of a person's body can be automatically changed over time by moving a lens, prism, or lightguide through which the light is transmitted.

In an example, the angle at which light is emitted from a light emitter leaves a device and/or the angle at which the light intersects the surface of a person's body can be automatically changed over time by moving a mirror from which the light is reflected. In an example, the angle at which light is emitted from a light emitter leaves a device and/or the angle at which the light intersects the surface of a person's body can be automatically changed over time in response to movement (e.g. shifting or rotation) of the device relative to the person's body.

In an example, a beam of light from a light emitter can be automatically moved by using an actuator to automatically rotate, tilt, raise, or lower a lens through which this light is transmitted. In an example, a beam of light emitted from a light emitter can be automatically moved by using an actuator to automatically change the focal distance of a lens through which this light is transmitted. In an example, the beam of light from a light emitter can be automatically moved by using an actuator to automatically move a light guide through which this light is transmitted.

In an example, a beam of light emitted by a light emitter can be automatically moved by using an actuator to automatically rotate, tilt, raise, or lower a light guide through which this light is transmitted. In an example, a beam of light emitted by a light emitter can be automatically moved by using an actuator to automatically move a light reflector (such as a mirror) from which this light is reflected. In an example, a beam of light emitted by a light emitter can be automatically moved by using an actuator to automatically rotate, tilt, raise, or lower a light reflector (such as a mirror) from which this light is reflected.

In an example, a wearable device can further comprise a track, channel, or slot along which a light emitter, a light receiver, or both can be moved. In an example, this movement can be done manually. In an example, this movement can be done automatically by one or more actuators. In an example, this track, channel, or slot can have a circumferential orientation. In an example, this track, channel, or slot can have a proximal-to-distal orientation. In an example, the distance between a light emitter and a light receiver can be adjusted by moving the light emitter, the light receiver, or both along such a track, channel, or slot. In an example, the location of a light emitter and/or a light receiver relative to a person's body can be adjusted by moving the light emitter, the light receiver, or both along such a track, channel, or slot.

In an example, movement of a light emitter, a light receiver, or both along a track, channel, or slot can enable customization of a device to the anatomy of a specific person for more accurate measurement of that person's biometric parameter. In an example, varying the distance between a light receiver and one or more light emitters at different times can measure analyte concentration at different tissue levels and locations in a person's finger, wrist, and/or arm.

In an example, a wearable device can include a rotating member which holds a light emitter, a light receiver, or both. In an example, rotation of this member can be done manually. In an example, this rotation can be done automatically by one or more actuators. In an example, the distance between a light emitter and a light receiver can be adjusted by rotating the rotating member. In an example, the location of a light emitter and/or a light receiver relative to a person's body can be adjusted by rotating the rotating member.

In an example, a selected geometric configuration of a plurality of light emitters and a light receiver can be designed to most accurately measure biometric parameter level. In an example, the geometric configuration of a plurality of light emitters and a light receiver can be adjusted automatically (in an iterative manner) by a device in order to more accurately measure biometric parameter level for a specific person, for a specific type of activity, or for a specific configuration of the device relative to the person's body surface.

In an example, the geometric configuration of a plurality of light emitters and a light receiver can be adjusted automatically to maintain accurate measurement of biometric parameter level even if the device shifts and/or moves relative to the person's body surface. In an example, a wearable device can automatically vary the geometric configuration of a plurality of light emitters and a light receiver in order to scan through a range of tissue depths, locations, and/or types in order to measure biometric parameter level more accurately.

In an example, a light emitter of this system can be automatically moved by an actuator relative to a housing which holds it. In an example, a light emitter can be automatically tilted by an actuator. In an example, a light emitter can be automatically rotated by an actuator. In an example, a light emitter can be automatically raised or lowered by an actuator. In an example, a light emitter can be automatically tilted, rotated, raised, or lowered when the wearable housing which holds it moves relative to the body surface on which it is worn. In an example, a light emitter can be automatically tilted, rotated, raised, or lowered in order to maintain a selected distance (or distance range) from the surface of a person's body. In an example, a light emitter can be automatically tilted, rotated, raised, or lowered in order to maintain a selected angle (or angle range) with respect to the surface of a person's body.

In an example, varying the distance between a light receiver and different light emitters at different times can measure analyte concentration at different tissue levels and locations in a person's finger, wrist, and/or arm. In an example, the depth, breadth, location, and/or type of body tissue or fluid through which light passes can be changed by adjusting the location or shape of a light emitter. In an example, the location or shape of a light emitter can be adjusted in order to more accurately measure biometric parameter level. In an example, the location or shape of a light emitter can be adjusted automatically (in an iterative manner) by a device in order to more accurately measure biometric parameter level for a specific person, for a specific type of activity, or for a specific configuration of the device relative to the person's body surface.

In an example, the location or shape of a light emitter can be adjusted automatically to maintain accurate measurement of biometric parameter level even if the device shifts and/or moves relative to the person's body surface. In an example, a wearable device can automatically vary the location a light emitter to scan through a range of tissue depths, locations, and/or types in order to obtain more accurate measurement of biometric parameter level.

In an example, the circumferential (e.g. polar, compass, or clock-hour) location of a light emitter on the circumference of the person's wrist, finger, or arm can be automatically changed over time by the device. In an example, the circumferential (e.g. polar, compass, or clock-hour) location of a light emitter on the circumference of the person's wrist, finger, or arm can be automatically shifted and/or moved by the device. In an example, a wearable device can have an arcuate channel, groove, or track (around at least part of its circumference) along which a light emitter can be automatically moved (e.g. rotated) by an actuator in the device.

In an example, a light emitter on a person's wrist, finger, or arm can be automatically moved (e.g. rotated) by the device from one circumferential quadrant of the person's wrist, finger, or arm to another circumferential quadrant of the person's wrist, finger, or arm. In an example, a light emitter can be moved (e.g. rotated) around at least half of the circumference of the device. In an example, the circumferential location of a light emitter on the circumference of the person's wrist, finger, or arm can be automatically oscillated and/or iteratively-varied by a device. In an example, the angle and/or vector of light which has been emitted from a light emitter can be automatically changed over time by movement of a micromirror, microprism, or microlens. In an example, the angle and/or vector of light which has been emitted from a light emitter can be automatically oscillated and/or iteratively-varied by the device.

In an example, there can be differences in the distance and/or pressure between different light emitters and a person's wrist, finger, or arm. In an example, there can be a first distance and/or pressure between a first light emitter in the array and a person's body surface and a second distance and/or pressure between a second light emitter in the array and the person's body surface. In an example, the distance and/or pressure between a light emitter relative to the person's body can be automatically changed over time by the device. In an example, the distance and/or pressure from a light emitter relative to the person's body can be automatically oscillated and/or iteratively-varied by the device.

In an example, a wearable device can include one or more light barriers (e.g. light shields, light blockers, cladding, and/or opaque barriers) between one or more light emitters and one or more light receivers which reduce the transmission of light energy directly from the light emitter to the light receiver (without intended light transmission through body tissue). In an example, a wearable device can include one or more light barriers (e.g. light shields, light blockers, cladding, and/or opaque barriers) between ambient light and one or more light receivers which reduce the transmission of light energy from the ambient light to the light receiver.

Direct transmission of light from a light emitter to a light receiver or transmission of ambient light to a light receiver can cause errors in the measurement of one or more biometric parameters. One or more light barriers (e.g. light shields, light blockers, cladding, and/or opaque barriers) can reduce or eliminate such errors. In an example, a light receiver can be optically isolated by a light barrier, shield, blocker, and/or cladding so that only light which reaches light receiver has been reflected from or transmitted through body tissue, organs, and/or fluid.

In an example, a light barrier can surround a perimeter around a light single receiver. In an example, a light barrier can surround a perimeter around a subset (e.g. two or more) of light receivers. In an example, a light barrier can surround a perimeter around a light single emitter. In an example, a light barrier can surround a perimeter around a subset (e.g. two or more) of light emitters.

In an example, each light receiver in a wearable device can be optically isolated from direct transmission (e.g. not through body tissue) of light from one or more light emitters by a separate light barrier. In an example, each light receiver in a wearable device can be optically isolated from direct transmission (e.g. not through body tissue) of light from one or more light emitters by a circular and/or annular light barrier. In an example, subsets of one or more light receivers can be optically isolated from direct transmission (e.g. not through body tissue) of light from one or more light emitters by the same light barrier. In an example, all light receivers in a wearable device can be optically isolated from direct transmission (e.g. not through body tissue) of light from one or more light emitters by the same light barrier.

In an example, each light receiver in a wearable device can be optically isolated from ambient light by a separate light barrier. In an example, each light receiver in a wearable device can be optically isolated from ambient light by a circular and/or annular light barrier. In an example, subsets of one or more light receivers can be optically isolated from ambient light by the same light barrier. In an example, all light receivers in a wearable device can be optically isolated from ambient light by the same light barrier.

In an example, there can be a circular and/or annular light barrier around each light receiver in a wearable device. In an example, a light barrier can encircle two or more light receivers. In an example, there can be one light barrier which encircles all light receivers in a wearable device. In an example, there can be a light barrier around each light emitter in a wearable device. In an example, there can be a circular and/or annular light barrier around each light emitter in a wearable device. In an example, a light barrier can encircle two or more light emitters. In an example, there can be one light barrier which encircles all light emitters in a wearable device.

In an example, there can be a light barrier around each light receiver in a wearable device. In an example, there can be a circular and/or annular light barrier around each light receiver in a wearable device. In an example, a light barrier can encircle two or more light receivers. In an example, there can be one light barrier which encircles all light receivers in a wearable device. In an example, there can be a light barrier around each light emitter in a wearable device. In an example, there can be a circular and/or annular light barrier around each light emitter in a wearable device. In an example, a light barrier can encircle two or more light emitters. In an example, there can be one light barrier which encircles all light emitters in a wearable device.

In an example, a light barrier can have a linear shape. In an example, a light barrier can have a polygonal (e.g. square, hexagonal, or octagonal) shape. In an example, a light barrier can have a circular, elliptical, or annular shape. In an example, a light barrier can have an undulating and/or sinusoidal shape. In an example, a light barrier can have a polygonal (e.g. square, hexagonal, or octagonal) shape, wherein a light receiver is at the center of this shape. In an example, a light barrier can have a circular, elliptical, or annular shape, wherein a light receiver is at the center of this shape. In an example, a light barrier can be an opaque ring around a light receiver. In an example, this light barrier can be an opaque partition between a light emitter and a light receiver. In an example, a light barrier can have a longitudinal axis which is substantially parallel to the vector of (collimated or coherent) light beams from a light emitter.

In an example, a light barrier between a light emitter and a light receiver can be opaque. In an example, a light barrier between a light emitter and a light receiver can be compressible, flexible, and/or elastic. In an example, a light barrier can be made from an opaque compressible polymer. In an example, a light barrier can be made from an opaque elastomeric polymer. In an example, a light barrier can be made from an opaque elastomeric silicone-based polymer. In an example, a light barrier can comprise compressible foam.

In an example, a wearable device can include a circular, annular, circumferential, and/or polygonal light barrier between (sets of) light emitters in the device and light receivers in the device so that light from light emitters only reaches light receivers after it has been reflected by or transmitted through body tissue. In an example, a wearable device can include a plurality of light barriers and a plurality of light receivers, wherein there is one light barrier for each light receiver. In an example, a light barrier can comprise a ring of opaque material between a light emitter and a light receiver (or set of light receivers). In an example, a light barrier can comprise a polygon (e.g. quadrilateral, hexagon, or octagon shaped member) of compressible, compliant, and/or elastomeric opaque material between ambient light and a light receiver (or set of light receivers).

In an example, a wearable device can include two circular, annular, circumferential, and/or polygonal light barriers: one light barrier between light emitters in the device and light receivers in the device; and one light barrier between ambient light and light receivers in the device. In an example, a light barrier can be made from opaque foam. In an example, a wearable device can include four light barriers and four light receivers, wherein there is one light barrier for each light receiver. In an example, a wearable device can include four light barriers and four sets of light receivers, wherein there is one light barrier for each light receiver. In an example, a light barrier can comprise a ring of compressible, compliant, and/or elastomeric opaque material between a light emitter and a light receiver (or set of light receivers). In an example, a light barrier can comprise a ring of opaque material between ambient light and a light receiver (or set of light receivers).

In an example a wearable device can comprise an alternating (emitter then receiver) array of (sets of) light emitters and light receivers, wherein each light receiver is surrounded by a light barrier which prevents light from a light emitter from reaching the light receiver without first being reflected from, or transmitted through, body tissue. In an example, a light barrier can comprise a circular and/or annular opaque ring between ambient light and a light receiver (or set of light receivers). In an example, a light barrier can comprise a polygon (e.g. quadrilateral, hexagon, or octagon shaped member) of opaque material between a light emitter and a light receiver (or set of light receivers). In an example a wearable device can comprise an alternating (emitter then receiver) array of (sets of) light emitters and light receivers, wherein light receivers are surrounded by a light barrier which prevents light from light emitters from reaching the light receivers without first being reflected from, or transmitted through, body tissue.

In an example, a light barrier can comprise a polygon (e.g. quadrilateral, hexagon, or octagon shaped member) of compressible, compliant, and/or elastomeric opaque material between a light emitter and a light receiver (or set of light receivers). In an example, a light barrier can comprise a ring of compressible, compliant, and/or elastomeric opaque material around a light receiver. In an example a wearable device can comprise an alternating (emitter then receiver) array of light emitters and light receivers, wherein each light receiver is surrounded by a light barrier which prevents light from a light emitter from reaching the light receiver without first being reflected from, or transmitted through, body tissue.

In an example, a wearable device can comprise: a housing (e.g. a smart watch housing or finger ring) which is worn on a person's wrist, arm, or finger; an array of light emitter sets and light receivers on the housing; wherein light emitted from the light emitters is reflected from and/or transmitted through the person's body tissue and received by the light receivers; wherein attributes (e.g. the spectral distribution) of light received by the light receivers are analyzed to measure one or more biometric parameters and/or detect one more physiological conditions of the person; wherein the array has a circular, approximately-circular polygonal (e.g. hexagonal or octagonal), annular, or circumferential shape; wherein there is an alternating pattern of light emitter sets and light receivers around the perimeter of the array; wherein there are a plurality (e.g. three or more) of light emitters in each light emitter set; wherein different light emitters in a set emit light with different colors, wavelengths, and/or frequencies; and wherein at least one light emitter in a set emits red light, at least one light emitter in a set emits infrared light, and at least one light emitter in a set emits green light.

In an example, a wearable device can comprise: (a) a housing (e.g. a smart watch housing or finger ring) which is worn on a person's wrist, arm, or finger; an array of light emitter sets and light receivers on the housing; wherein light emitted from the light emitters is reflected from and/or transmitted through the person's body tissue and received by the light receivers; wherein attributes (e.g. the spectral distribution) of light received by the light receivers are analyzed to measure one or more biometric parameters and/or detect one more physiological conditions of the person; wherein the array has a circular, approximately-circular polygonal (e.g. hexagonal or octagonal), annular, or circumferential shape; wherein there is an alternating pattern of light emitter sets and light receivers around the perimeter of the array; wherein there are a plurality (e.g. three or more) of light emitters in each light emitter set; wherein different light emitters in a set emit light with different colors, wavelengths, and/or frequencies; and wherein at least one light emitter in a set emits red light, at least one light emitter in a set emits infrared light, and at least one light emitter in a set emits green light; and (b) one or more (compressible, compliant, and/or elastomeric) opaque light barriers surrounding one or more light receivers; wherein the one or more light barriers reduce or eliminate transmission of light from the light emitters to the light receivers which has not been reflected by or transmitted through the person's body tissue; and/or wherein the one or more light barriers reduce or eliminate transmission of ambient light to the light emitters.

In an example, a wearable device can comprise: (a) a housing (e.g. a smart watch housing or finger ring) which is worn on a person's wrist, arm, or finger; an array of light emitter sets and light receivers on the housing; wherein light emitted from the light emitters is reflected from and/or transmitted through the person's body tissue and received by the light receivers; wherein attributes (e.g. the spectral distribution) of light received by the light receivers are analyzed to measure one or more biometric parameters and/or detect one more physiological conditions of the person; wherein the array has a circular, approximately-circular polygonal (e.g. hexagonal or octagonal), annular, or circumferential shape; wherein there is an alternating pattern of light emitter sets and light receivers around the perimeter of the array; wherein there are a plurality (e.g. three or more) of light emitters in each light emitter set; wherein different light emitters in a set emit light with different colors, wavelengths, and/or frequencies; and wherein at least one light emitter in a set emits red light, at least one light emitter in a set emits infrared light, and at least one light emitter in a set emits green light; and (b) one or more compressible, compliant, and/or elastomeric opaque light barriers surrounding one or more light receivers; wherein the one or more light barriers reduce or eliminate transmission of light from the light emitters to the light receivers which has not been reflected by or transmitted through the person's body tissue; and/or wherein the one or more light barriers reduce or eliminate transmission of ambient light to the light emitters.

In an example, a wearable device can include a plurality of light barriers and a plurality of light receivers, wherein each light barrier surrounds a subset of light receivers. In an example, a wearable device can include a plurality of light barriers and a plurality of light receivers, wherein there is one light barrier around (e.g. surrounding) each light receiver. In an example, a light barrier can comprise a polygon (e.g. quadrilateral, hexagon, or octagon shaped member) of opaque material around a light receiver. In an example a wearable device can comprise an alternating (emitter then receiver) array of light emitters and light receivers, wherein light receivers are surrounded by a light barrier which prevents light from light emitters from reaching the light receivers without first being reflected from, or transmitted through, body tissue.

In an example, a light barrier can comprise a polygon (e.g. quadrilateral, hexagon, or octagon shaped member) of compressible, compliant, and/or elastomeric opaque material around a light receiver. In an example, a light barrier can be made from an opaque material. In an example, a wearable device can include eight light barriers and eight light receivers, wherein there is one light barrier for each light receiver. In an example, a light barrier can be made from opaque ink. In an example, a light barrier can comprise a circular and/or annular opaque ring between a light emitter and a light receiver (or set of light receivers). In an example, a wearable device can include two light barriers: one light barrier between (sets of) light emitters in the device and light receivers in the device; and one light barrier between ambient light and light receivers in the device.

In an example, a light barrier can form a perimeter around a light receiver. In an example, a wearable device can include a circular, annular, circumferential, and/or polygonal light barrier between light emitters in the device and light receivers in the device so that light from light emitters only reaches light receivers after it has been reflected by or transmitted through body tissue. In an example, a light barrier can comprise a ring of opaque material around a light receiver. In an example, a wearable device can include two circular, annular, circumferential, and/or polygonal light barriers: one light barrier between light emitters in the device and light receivers in the device; and one light barrier between ambient light and light receivers in the device.

In an example, a light barrier can be made from a compressible, compliant, and/or elastomeric opaque material. In an example, a wearable device can include six light barriers and six light receivers, wherein there is one light barrier for each light receiver. In an example, a light barrier can comprise a polygon (e.g. quadrilateral, hexagon, or octagon shaped member) of opaque material between ambient light and a light receiver (or set of light receivers). In an example, a light barrier can comprise a circular and/or annular opaque ring around a light receiver. In an example, a wearable device can include two light barriers: one light barrier between light emitters in the device and light receivers in the device; and one light barrier between ambient light and light receivers in the device. In an example, a light barrier can comprise a ring of compressible, compliant, and/or elastomeric opaque material between ambient light and a light receiver (or set of light receivers).

In an example, a wearable device can comprise: (a) a housing (e.g. a smart watch housing or finger ring) which is worn on a person's wrist, arm, or finger; an array of light emitter sets and light receivers on the housing; wherein light emitted from the light emitters is reflected from and/or transmitted through the person's body tissue and received by the light receivers; wherein attributes (e.g. the spectral distribution) of light received by the light receivers are analyzed to measure one or more biometric parameters and/or detect one more physiological conditions of the person; wherein the array has a circular, approximately-circular polygonal (e.g. hexagonal or octagonal), annular, or circumferential shape; wherein there is an alternating pattern of light emitter sets and light receivers around the perimeter of the array; wherein there are a plurality (e.g. three or more) of light emitters in each light emitter set; wherein different light emitters in a set emit light with different colors, wavelengths, and/or frequencies; and wherein at least one light emitter in a set emits red light, at least one light emitter in a set emits infrared light, and at least one light emitter in a set emits green light; and (b) one or more circular, annular, and/or circumferential opaque light barriers surrounding one or more light receivers; wherein the one or more light barriers reduce or eliminate transmission of light from the light emitters to the light receivers which has not been reflected by or transmitted through the person's body tissue; and/or wherein the one or more light barriers reduce or eliminate transmission of ambient light to the light emitters.

Light from a light emitter which is received by a light receiver after the light has been reflected from and/or transmitted through body tissue (or fluid) can be analyzed to determine how the spectral distribution of that light has been changed by interaction with the body tissue (or fluid). In an example, different types of chemicals, molecules, and tissue structures can absorb different portions of a light spectrum. In an example, changes in the spectral distribution of this light caused by this differential absorption can, in turn, be used to measure one or more biometric parameters. In an example, biometric parameters which are measured can be selected from the group consisting of: body oxygenation, heart rate, heart rate variability, body hydration level, and blood glucose level.

Analysis of these spectral changes can be called “spectroscopic analysis” or “spectral analysis”. A combination of one or more light emitters and one or more light receivers used for such analysis can be called a “spectroscopic sensor” or “spectroscopy sensor.” In an example, one or more light emitters and one or more light receivers can comprise a spectroscopic sensor selected from the group consisting of: backscattering spectrometry sensor, infrared spectroscopy sensor, ion mobility spectroscopic sensor, mass spectrometry sensor, Near Infrared spectroscopy sensor (NIS), Raman spectroscopy sensor, spectrometry sensor, spectrophotometer, spectroscopy sensor, ultraviolet spectroscopy sensor, and white light spectroscopy sensor.

In an example, a wearable device can be configured to receive light which has been reflected from or transmitted through body tissue, organs, and/or fluid selected from the group consisting of: blood, blood vessels, body fat, brain tissue, dermis, ear drum, earlobe, epidermis, fat tissue, intercellular fluid, lymphatic fluid, lymphatic passageways, muscle tissue, nerve tissue, saliva, skin, sweat, and tears. In an example, portions of the spectrum of light emitted by a light emitter can be absorbed by body tissue, organs, and/or fluid. In an example, spectroscopic analysis of these absorbed portions can enable measurement of a biometric parameter. In an example, portions of the spectrum of light emitted by a light emitter can be shifted by interaction with body tissue, organs, and/or fluid. In an example, spectroscopic analysis of these shifted portions can enable measurement of a biometric parameter.

In an example, changes, gaps, and/or shifts in selected frequencies in the spectral distribution of light caused by interaction with a person's body tissue and/or fluid can be analyzed to monitor changes in the chemical composition of the person's body tissue and/or fluid. In an example, data from a spectroscopic sensor can be analyzed to determine how the spectral distribution of light has been changed by reflection from, or passage through, body tissue, organs, and/or fluid. This, in turn, can be used to estimate the values of one or more biometric parameters and/or identify one or more physiological conditions.

In an example, changes in a person's biometric parameter can cause changes in the volume and flow of blood through the person's wrist, arm, and/or finger. In an example, a wearable device can collect data from an array of spectroscopic sensors (e.g. light emitters and light receivers) concerning changes in the volume and/or flow of the person's blood which can, in turn, be used to estimate changes in a person's biometric parameter. In an example, a wearable device can include a photoplethysmography (PPG) sensor. In an example, changes in a person's biometric parameter can cause changes in the volume of interstitial fluid in the person's wrist, arm, and/or finger. In an example, a wearable device can collect data from an array of spectroscopic sensors (e.g. light emitters and light receivers) concerning changes in the volume of the person's interstitial fluid which can, in turn, be used to estimate changes in the person's biometric parameter.

In an example, portions of the spectrum of light emitted by a light emitter can be absorbed by body tissue and spectral analysis of these absorbed portions can enable measurement of body oxygenation, heart rate, heart rate variability, body hydration level, and/or blood glucose level. In an example, portions of the spectrum of light emitted by a light emitter can be shifted by interaction with body tissue and spectral analysis of these shifted portions can enable measurement of body oxygenation, heart rate, heart rate variability, body hydration level, and/or blood glucose level.

In an example, a wearable device can comprise one or more near-infrared light spectroscopic sensors. In an example, a wearable device can comprise one or more ultraviolet light spectroscopic sensors. In an example, a wearable device can have an array of spectroscopic sensors which are distributed around the radially-inward-facing circumference of the device. In an example, spectroscopic sensors can be evenly distributed along different locations around the circumference of a wearable device. In an example, spectroscopic sensors can be clustered on ventral and/or dorsal portions of the circumference of a wearable device. In another example, an array of spectroscopic sensors can be distributed around the radially-inward-facing side of a modular smart watch strap, a specialized wrist-worn band, or a finger ring.

In an example, a spectroscopic sensor can be selected from the group consisting of: spectroscopic sensor, spectrometry sensor, white light spectroscopic sensor, infrared spectroscopic sensor, near-infrared spectroscopic sensor, ultraviolet spectroscopic sensor, ion mobility spectroscopic sensor, mass spectrometry sensor, backscattering spectrometry sensor, and spectrophotometer. In an example, a spectroscopic sensor can be selected from the group consisting of: ambient light spectroscopic sensor, analytical chromatographic sensor, backscattering spectrometry sensor, spectroscopic camera, chemiluminescence sensor, chromatographic sensor, coherent light spectroscopic sensor, colorimetric sensor, fiber optic spectroscopic sensor, fluorescence sensor, gas chromatography sensor, infrared light sensor, infrared spectroscopic sensor, ion mobility spectroscopic sensor, laser spectroscopic sensor, liquid chromatography sensor, mass spectrometry sensor, near infrared spectroscopic sensor, optoelectronic sensor, photocell, photochemical sensor, photodetector, photoplethysmography (PPG) sensor, Raman spectroscopic sensor, spectral analysis sensor, spectrographic sensor, spectrometric sensor, spectrometry sensor, spectrophotometer, spectroscopic body hydration sensor, spectroscopic oximeter, ultraviolet light sensor, ultraviolet spectroscopic sensor, visible light spectroscopic sensor, and white light spectroscopic sensor.

In an example, a wearable device can comprise: an attachment member which spans at least a portion of the circumference of a person's wrist (and/or arm); an enclosure (or housing) which is part of and/or attached to the attachment member; a first spectroscopic sensor in the enclosure (or housing) which projects a beam of light onto the wrist (and/or arm) surface at a first angle relative to the enclosure; and a second spectroscopic sensor in the enclosure which projects a beam of light onto the wrist (and/or arm) surface at a second angle relative to the enclosure (or housing), wherein the first angle differs from the second angle by at least 10 degrees.

In an example, a spectroscopic sensor (e.g. spectrometer) of a wearable device can comprise a light emitter which emits near infrared light. In an example, a spectroscopic sensor of a wearable device can comprise a light emitter which emits infrared light. In an example, a light emitter can emit light with a wavelength in the range of 400 to 700 nanometers. In an example, a light emitter can emit light with a wavelength in the range of 300 to 1200 nanometers. In an example, a wearable device can comprise one or more spectroscopic sensors selected from the group consisting of: near-infrared spectroscopic sensor; infrared spectroscopic sensor; white light spectroscopic sensor; and ultraviolet spectroscopic sensor. In an example, one or more light emitters can be selected from the group consisting of: white LED, blue LED, red LED, infrared LED, and green LED.

In an example, a spectroscopic sensor (e.g. spectrometer) can comprise a plurality of light emitters which emit light at different times. In an example, a spectroscopic sensor can comprise an array of light emitters which emit light pulses at different times. In an example, a spectroscopic sensor can comprise a linear array of light emitters which emit light pulses at different times. In an example, a spectroscopic sensor can comprise an annular array of light emitters which emit light pulses at different times. In an example, a spectroscopic sensor can comprise a plurality of light emitters which are selectively and sequentially activated. In an example, a plurality of light emitters can be selectively and sequentially activated via time-based multiplexing. In an example, a spectroscopic sensor can operate with time-based multiplexing.

In an example, a spectroscopic sensor (e.g. spectrometer) can comprise an array with a plurality of light emitters and a plurality of light receivers. In an example, a spectroscopic sensor can comprise one light emitter and two light receivers. In an example, a spectroscopic sensor can comprise two light emitters and one light receiver. In an example, a spectroscopic sensor can comprise a plurality of light emitters at different locations. In an example, a spectroscopic sensor can comprise a two-dimensional arcuate array with at least one light emitter and at least one light receiver. In an example, a spectroscopic sensor can comprise a three-dimensional array of light emitters and receivers. In an example, a spectroscopic sensor can comprise a plurality of light emitters and receivers in a three-dimensional matrix or grid. In an example, a spectroscopic sensor can comprise a plurality of light emitters which emit light at different angles.

In an example, a spectroscopic sensor (e.g. spectrometer) can comprise a circular or annular array with at least one light emitter and at least one light receiver. In an example, a spectroscopic sensor can comprise a ring of light emitters and receivers. In an example, a spectroscopic sensor can comprise a plurality of light emitters in a ring or circle around a light receiver. In an example, a spectroscopic sensor can comprise at least one light emitter and at least one light receiver in a concentric configuration. In an example, a spectroscopic sensor can comprise a plurality of light emitters in a polygonal configuration around a light receiver. In an example, a spectroscopic sensor can comprise a polygonal array with at least one light emitter and at least one light receiver.

In an example, a wearable device which uses spectroscopic analysis to measure one or more biometric parameters can include one or more mirrors in addition to light emitters and light receivers. In an example, a wearable device which uses spectroscopic analysis to measure one or more biometric parameters can include a digital micromirror device in addition to light emitters and light receivers. In an example, a wearable device which uses spectroscopic analysis to measure one or more biometric parameters can include a micromirror array in addition to light emitters and light receivers.

In an example, a wearable device which uses spectroscopic analysis to measure one or more biometric parameters can include one or more micromirrors which change the vectors of light beams emitted from light emitters. In an example, a wearable device which uses spectroscopic analysis to measure one or more biometric parameters can include one or more movable micromirrors which change the vectors of light beams emitted from light emitters. In an example, a wearable device which uses spectroscopic analysis to measure one or more biometric parameters can include one or more rotating and/or pivoting micromirrors which change the vectors of light beams emitted from light emitters.

In an example, a wearable device which uses spectroscopic analysis to measure one or more biometric parameters can include one or more light guides in addition to light emitters and light receivers. In an example, a wearable device which uses spectroscopic analysis to measure one or more biometric parameters can include one or more wave guides in addition to light emitters and light receivers. In an example, a wearable device which uses spectroscopic analysis to measure one or more biometric parameters can include one or more optical fibers in addition to light emitters and light receivers. In an example, a wearable device which uses spectroscopic analysis to measure one or more biometric parameters can include one or more light guides, wave guides, and/or optical fibers which change the vectors of light beams emitted from light emitters.

In an example, a wearable device can include one or more light guides, wave guides, and/or optical fibers which direct light from a first location, angle, and/or transmission vector to a second location, angle, and/or transmission vector. In an example, a light guide, wave guide, or optical fiber can direct light from a light emitter toward body tissue, organs, and/or fluid. In an example, a light guide, wave guide, or optical fiber can direct light reflected from, or having passed through, body tissue, organs, and/or fluid toward a light receiver. In an example, one or more light emitters can deliver light to body tissue, organs, and/or fluid indirectly via one or more light guides, wave guides, and/or optical fibers.

In an example, a spectroscopic sensor (e.g. spectrometer) can further comprise an optical filter. In an example, a spectroscopic sensor can further comprise two-dimensional array of optical filters. In an example, a spectroscopic sensor can further comprise one or more optical filters selected from the group consisting of: optical absorption filter; acousto-optic filter; Bragg filter; cascaded filter; dielectric thin-film filter; Fabry-Perot filter; hybrid filter; and optical interference filter. In an example, a spectroscopic sensor can further comprise a lens array.

In an example, a wearable device can include a battery or other energy source. In an example, a battery or other energy source can power one or more light emitters. In an example, a wearable device can further comprise an energy source which powers a light emitter, a light receiver, a data processor, and/or a data transmitter. In an example, a wearable device can include an energy source which powers a light emitter, a light receiver, a data processor, a data transmitter, and/or a charge coupled device. In an example, an energy source can transduce, harvest, and/or generate energy from body motion or kinetic energy. In an example, an energy source can transduce, harvest, and/or generate energy from ambient (e.g. solar) light. In an example, an energy source can transduce, harvest, and/or generate energy from body thermal energy. In an example, an energy source can transduce, harvest, and/or generate energy from ambient electromagnetic energy.

In an example, a wearable device can include a data processor (e.g. central processing unit, microchip, and/or microprocessor). In an example, a wearable device can include a local data processor. In an example, a wearable device can include data transmitter and/or data receiver. In an example, a wearable device can include wireless data transmitter and/or wireless data receiver. In an example, a wearable device can be part of a system that includes data transmitter which is in electronic communication with a remote data processor. In an example, a wearable device can be in wireless communication with an external device selected from the group consisting of: a cell phone, an electronic tablet, electronically-functional eyewear, a home electronics portal, an implanted medical device, an internet portal, a laptop computer, a mobile computer, a mobile phone, a remote computer, a remote control unit, a smart phone, a smart utensil, a television set, and a wearable data processing hub. In an example, additional data processing and analysis can be done within an external device.

In an example, a wearable device or system can further include a computer-to-human interface which is selected from the group consisting of: auditory feedback (such as a voice message, alarm, buzzer, ring tone, or song); feedback via computer-generated speech; mild external electric charge or neural stimulation; periodic feedback at a selected time of the day or week; phantom taste or smell; phone call; pre-recorded audio or video message by the person from an earlier time; television-based messages; and tactile, vibratory, or pressure-based feedback.

In an example, a wearable device or system can further comprise a data processing unit or microprocessor. In an example, a wearable device or system can further comprise a battery, energy transducer, or other power source. In an example, a wearable device or system can further comprise a watch band, watch face, and/or watch strap. In an example, a wearable device or system can further comprise a display screen or touch screen. In an example, a wearable device or system can further comprise a finger ring. In an example, a wearable device or system can further comprise a fitness band.

In an example, a wearable device or system can be in wireless communication with an external device. In an example, a wearable device can be in wireless communication with an external device selected from the group consisting of: a cell phone, an electronic tablet, electronically-functional eyewear, a home electronics portal, an internet portal, a laptop computer, a mobile phone, a remote computer, a remote control unit, a smart phone, a smart utensil, a television set, and a virtual menu system.

In an example, a wearable device or system can further comprise a augmented reality eyewear and/or smart eyewear. In an example, a wearable device or system can further comprise a cell phone and/or mobile phone. In an example, a wearable device or system can further comprise a desktop computer, electronic tablet, or laptop. In an example, a wearable device or system can further comprise a keypad or buttons. In an example, a wearable device or system can further comprise a camera. In an example, a wearable device or system can further comprise a chemical sensor, electromagnetic sensor, microphone, motion sensor, optical sensor, or photoplethysmography (PPG) sensor.

In an example, a wearable device or system can further comprise one or more other types of biometric or environmental sensors in addition to the primary light emitters and light receivers. In an example, a wearable device or system can also include a non-light energy emitter and a non-light energy receiver. In an example, a wearable device can comprise light and electromagnetic energy sensors for measuring body oxygenation, heart rate, heart rate variability, body hydration level, and/or blood glucose level. In an example, a wearable device can comprise spectroscopic and microwave energy sensors for measuring body oxygenation, heart rate, heart rate variability, body hydration level, and/or blood glucose level.

In an example, a wearable device or system can further comprise one or more sensors selected from the group consisting of: electrocardiographic (ECG) sensor, strain gauge, magnetometer, inertial sensor, electromagnetic sensor, sweat sensor, biochemical sensor, photochemical sensor, and electromyographic (EMG) sensor. In an example, a wearable device or system can further comprise one or more sensors selected from the group consisting of: thermal energy sensor, electroporation sensor, thermistor, galvanic skin response (GSR) sensor, tissue impedance sensor, chemoreceptor sensor, pulmonary function sensor, and gyroscope.

In an example, a wearable device or system can further comprise one or more sensors selected from the group consisting of: ultrasonic sensor, Hall-effect sensor, variable impedance sensor, humidity sensor, variable resistance sensor, motion sensor, microphone, conductivity sensor, and skin conductance sensor. In an example, a wearable device or system can further comprise one or more sensors selected from the group consisting of: electrical resistance sensor, skin moisture sensor, accelerometer, neurosensor, blood pressure sensor, piezocapacitive sensor, electromagnetic resistance sensor, and stretch sensor.

In an example, a wearable device or system can further comprise one or more sensors selected from the group consisting of: camera, piezoelectric sensor, chemiluminescence sensor, pressure sensor, magnetic field sensor, ballistocardiographic sensor, pH level sensor, and inclinometer. In an example, a wearable device or system can further comprise one or more sensors selected from the group consisting of: capacitance hygrometry sensor, piezoresistive sensor, impedance sensor, and variable translucence sensor.

In an example, data from a light receiver can be analyzed to measure body oxygenation, heart rate, heart rate variability, body hydration level, and/or blood glucose level using one or more methods selected from the group consisting of: Analysis of Variance (ANOVA), Artificial Neural Network (ANN), Auto-Regressive (AR) Modeling, Bayesian Analysis, Bonferroni Analysis (BA), Carlavian Curve Analysis (CCA), Centroid Analysis, Chi-Squared Analysis, Cluster Analysis, Correlation, Covariance, and Data Normalization (DN).

In an example, data from a light receiver can be analyzed to measure body oxygenation, heart rate, heart rate variability, body hydration level, and/or blood glucose level using one or more methods selected from the group consisting of: Decision Tree Analysis (DTA), Discrete Fourier transform (DFT), Discriminant Analysis (DA), Empirical Mode Decomposition (EMD), Factor Analysis (FA), Fast Fourier Transform (FFT), Feature Vector Analysis (FVA), and Fisher Linear Discriminant.

In an example, data from a light receiver can be analyzed to measure body oxygenation, heart rate, heart rate variability, body hydration level, and/or blood glucose level using one or more methods selected from the group consisting of: Fourier Transformation (FT) Method, Fuzzy Logic (FL) Modeling, Gaussian Model (GM), Generalized Auto-Regressive Conditional Heteroscedasticity (GARCH) Modeling, Hidden Markov Model (HMM), and Independent Components Analysis (ICA).

In an example, data from a light receiver can be analyzed to measure body oxygenation, heart rate, heart rate variability, body hydration level, and/or blood glucose level using one or more methods selected from the group consisting of: Inter-Band Power Ratio, Inter-Channel Power Ratio, Inter-Montage Power Mean, Inter-Montage Ratio, Kalman Filter (KF), Kernel Estimation, Laplacian Filter, Laplacian Montage Analysis, Least Squares Estimation, Linear Regression, Linear Transform, and Logit Model.

In an example, data from a light receiver can be analyzed to measure body oxygenation, heart rate, heart rate variability, body hydration level, and/or blood glucose level using one or more methods selected from the group consisting of: Machine Learning (ML), Markov Model, Maximum Entropy Modeling, Maximum Likelihood, Mean Power, Multi-Band Covariance Analysis, Multi-Channel Covariance Analysis, Multivariate Linear Regression, Multivariate Logit, Multivariate Regression, Naive Bayes Classifier, Neural Network, Non-Linear Programming, and Non-negative Matrix Factorization (NMF).

In an example, data from a light receiver can be analyzed to measure body oxygenation, heart rate, heart rate variability, body hydration level, and/or blood glucose level using one or more methods selected from the group consisting of: Power Spectral Density, Power Spectrum Analysis, Principal components Analysis (PCA), Probit Model, Quadratic Minimum Distance Classifier, Random Forest (RF), Random Forest Analysis (RFA), Regression Model, and Signal Amplitude (SA).

In an example, data from a light receiver can be analyzed to measure body oxygenation, heart rate, heart rate variability, body hydration level, and/or blood glucose level using one or more methods selected from the group consisting of: Signal Averaging, Signal Decomposition, Sine Wave Compositing, Singular Value Decomposition (SVD), Spine Function, Support Vector and/or Machine (SVM), Time Domain Analysis, Time Frequency Analysis, Time Series Model, Trained Bayes Classifier, Variance, Waveform Identification, Wavelet Analysis, and Wavelet Transformation.

In an example, a wearable device with light emitters and receivers for measuring a biometric parameter can be a band or strap. In an example, the band or strap can be elastic and/or flexible. In an example, the band or strap can be a stand-along band (e.g. fitness band). In an example, the band or strap can be a watch band or strap which is used with a watch housing (with a watch face). In an example, there can be light emitters and light receivers on both the housing of a smart watch and on the band or strap of the watch.

In an example, a wearable device with light emitters and receivers for measuring a biometric parameter can be a bracelet, bangle, armlet, or armband. In an example, a wearable device with light emitters and receivers for measuring a biometric parameter can be a finger ring. In an example, a wearable device with light emitters and receivers for measuring a biometric parameter can be incorporated into a shirt cuff or sleeve. In an example, a wearable device with light emitters and receivers for measuring a biometric parameter can be incorporated into a sock or leg band.

Having completed an introductory section which discussed general concepts, components, and methods which comprise this invention, this disclosure now provides descriptions for the specific examples which are shown in FIGS. 1 through 6.

FIG. 1 shows an example of a wearable device (e.g. smart watch or band) with: a housing that is attached to a wearable arcuate band; and a plurality of light energy emitters and a central light energy receiver which are held by this housing. In this example, light energy emitters encircle the light energy receiver. In this example, light energy emitters are arranged in an (approximately-circular) polygonal array around a central energy receiver. In an example, the housing and band can comprise a smart watch or band. In an example, the light energy emitters and light energy receiver can be located on the body-facing side of the housing. In an example, the opposite side of the housing can comprise a display and/or screen.

Specifically, FIG. 1 shows an oblique-side-perspective view of a wearable device (e.g. smart watch or band) comprising: an arcuate band 101 which is configured to span (some or all of) the circumferential perimeter of a part of a person's body (such as a wrist, arm, finger, ankle, and/or leg); a housing 109 which is attached to the arcuate band; a plurality of light energy emitters (106, 107, 108, 111, 112, 113, 114, and 115) which are held by the housing and configured to emit light energy toward the part of the person's body; a central light energy receiver 110 which is held by the housing and configured to receive light energy from the light energy emitters; a data processor 104 which is held by the housing and receives data from the light energy receiver; an energy source (e.g. battery) 103 which is held by the housing and provides energy to the light energy emitters and/or to the data processor; and a data transmitter 105 which is held by the housing and transmits data from the data processor to a remote device and/or remote location. This example further comprises another (type of) biometric or environmental sensor 102.

In an example, a wearable device can collect data from an annular array of light emitters and receivers. In an example, a sensor can comprise a ring of light emitters and receivers. In an example, a sensor can comprise a polygonal array with at least one light emitter and at least one light receiver. In an example, data from biometric sensors can be analyzed to measure a person's oxygen level. In an example, a ring of biometric sensors can collect data which is used to measure a person's blood and/or tissue oxygenation.

In an example, a wearable device can comprise a circular, annular, and/or octagonal array of light emitters and/or light receivers. In an example, a wearable device (e.g. a smart watch or band) can comprise a circular, annular, and/or octagonal array of alternating light emitters and light receivers. In an example, a device can have an annular array of four light emitters. In an example, a device can have an annular array of four light receivers. In an example, a device can have an annular array of four light emitters, and four light receivers. In an example, light emitters in an annular array can have polar coordinates which differ by 60 or 90 degrees. In an example, light receivers in an annular array can have polar coordinates which differ by 60 or 90 degrees.

In an example, a wearable device can comprise an array of sets of light emitters and light receivers. In an example, different sets can be activated at different times. In an example, there can be multiple triads of energy emitters and energy receivers. In an example, a wearable ring of biosensors can comprise at least three light emitters which emit light energy of different colors. In an example, a wearable ring of biosensors can comprise at least three light emitters which emit light energy at different wavelengths. In an example, one light emitter can emit red light, a second light emitter can emit infrared light, and a third light emitter can emit green light.

In example, a wearable device can comprise: a housing that is attached to a wearable arcuate band; and a plurality of energy emitters and central energy receiver which are held by this housing. In an example, energy emitters can encircle the energy receiver. In an example, energy emitters can be arranged in an (approximately-circular) polygonal array around the central energy receiver. In an example, the housing and band can comprise a smart watch, fitness band, or wearable hydration monitor. In an example, the energy emitters and energy receiver can be located on the inward-facing (body-facing) side of a housing. In an example, the opposite (outward-facing) side of a housing can comprise a computer display and/or screen.

In an example, a central energy receiver can be centrally located with respect to the energy emitters. In an example, there can be eight energy emitters around the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

In example, a wearable device can comprise: an arcuate band which is configured to span (some or all of) the circumferential perimeter of a part of a person's body (such as a wrist, arm, finger, ankle, and/or leg); a housing which is attached to the arcuate band; a plurality of energy emitters which are held by the housing and configured to emit energy toward the part of the person's body; a central energy receiver which is held by the housing and configured to receive energy from the energy emitters, wherein the plurality of energy emitters are around the central energy receiver (in an approximately-circular polygonal array); a data processor which is held by the housing and receives data from the energy receiver which is analyzed in order to measure the person's body hydration level; an energy source which is held by the housing and provides energy to the energy emitters and/or to the data processor; and a data transmitter which is held by the housing and transmits data from the data processor to a remote device and/or remote location.

In an example, a wearable device can be a smart watch housing, smart watch band, or other type of wearable band which is used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level

In an example, a wearable device can comprise an (approximately-circular) polygonal array of three sets of red, infrared, and green light emitters and three light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of three sets of red, infrared, and green light emitters and three light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level. Other example variations and component elaborations discussed elsewhere in this disclosure (or in other disclosures within the priority-linked family) can also be applied to this example.

FIG. 2 shows an example of a wearable device (e.g. smart watch or band) with: a housing that is attached to a wearable arcuate band; and a plurality of light energy emitters and light energy receiver receivers which are held by this housing. In this example, the wearable device comprises a circular and/or annular array of alternating light emitters and light receivers. In an example, the housing and band can comprise a smart watch or band. In an example, the light energy emitters and light energy receivers are located on the body-facing side of the housing. In an example, the opposite side of the housing comprises a display and/or screen.

Specifically, FIG. 2 shows an oblique-side-perspective view of a wearable device (e.g. smart watch or band) comprising: an arcuate band 201 which is configured to span (some or all of) the circumferential perimeter of a part of a person's body (such as a wrist, arm, finger, ankle, and/or leg); a housing 209 which is attached to the arcuate band; a plurality of light energy emitters and light energy receivers (206, 207, 208, 211, 212, 213, 214, and 215) which are held by the housing and configured to emit light energy toward the part of the person's body in an approximately-circular polygonal array; a data processor 204 which is held by the housing and receives data from the light energy receivers; an energy source 203 which is held by the housing and provides energy to the light energy emitters and/or to the data processor; and a data transmitter 205 which is held by the housing and transmits data from the data processor to a remote device and/or remote location. This example further comprises another (type of) biometric or environmental sensor 202.

In an example, a wearable device can collect data from an annular array of light emitters and receivers. In an example, a sensor can comprise a ring of light emitters and receivers. In an example, a sensor can comprise a polygonal array with at least one light emitter and at least one light receiver. In an example, data from biometric sensors can be analyzed to measure a person's oxygen level. In an example, a ring of biometric sensors can collect data which is used to measure a person's blood and/or tissue oxygenation.

In an example, a wearable device can comprise a circular, annular, and/or octagonal array of light emitters and/or light receivers. In an example, a wearable device (e.g. a smart watch or band) can comprise a circular, annular, and/or octagonal array of alternating light emitters and light receivers. In an example, a device can have an annular array of four light emitters. In an example, a device can have an annular array of four light receivers. In an example, a device can have an annular array of four light emitters, and four light receivers. In an example, light emitters in an annular array can have polar coordinates which differ by 60 or 90 degrees. In an example, light receivers in an annular array can have polar coordinates which differ by 60 or 90 degrees.

In an example, a wearable device can comprise an array of sets of light emitters and light receivers. In an example, different sets can be activated at different times. In an example, there can be multiple triads of energy emitters and energy receivers. In an example, a wearable ring of biosensors can comprise at least three light emitters which emit light energy of different colors. In an example, a wearable ring of biosensors can comprise at least three light emitters which emit light energy at different wavelengths. In an example, one light emitter can emit red light, a second light emitter can emit infrared light, and a third light emitter can emit green light.

In example, a wearable device can comprise: a housing that is attached to a wearable arcuate band; and a plurality of energy emitters and central energy receiver which are held by this housing. In an example, energy emitters can encircle the energy receiver. In an example, energy emitters can be arranged in an (approximately-circular) polygonal array around the central energy receiver. In an example, the housing and band can comprise a smart watch, fitness band, or wearable hydration monitor. In an example, the energy emitters and energy receiver can be located on the inward-facing (body-facing) side of a housing. In an example, the opposite (outward-facing) side of a housing can comprise a computer display and/or screen.

In an example, a central energy receiver can be centrally located with respect to the energy emitters. In an example, there can be eight energy emitters around the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

In example, a wearable device can comprise: an arcuate band which is configured to span (some or all of) the circumferential perimeter of a part of a person's body (such as a wrist, arm, finger, ankle, and/or leg); a housing which is attached to the arcuate band; a plurality of energy emitters which are held by the housing and configured to emit energy toward the part of the person's body; a central energy receiver which is held by the housing and configured to receive energy from the energy emitters, wherein the plurality of energy emitters are around the central energy receiver (in an approximately-circular polygonal array); a data processor which is held by the housing and receives data from the energy receiver which is analyzed in order to measure the person's body hydration level; an energy source which is held by the housing and provides energy to the energy emitters and/or to the data processor; and a data transmitter which is held by the housing and transmits data from the data processor to a remote device and/or remote location.

In an example, a wearable device can be a smart watch housing, smart watch band, or other type of wearable band which is used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level

In an example, a wearable device can comprise an (approximately-circular) polygonal array of three sets of red, infrared, and green light emitters and three light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of three sets of red, infrared, and green light emitters and three light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level. Other example variations and component elaborations discussed elsewhere in this disclosure (or in other disclosures within the priority-linked family) can also be applied to this example.

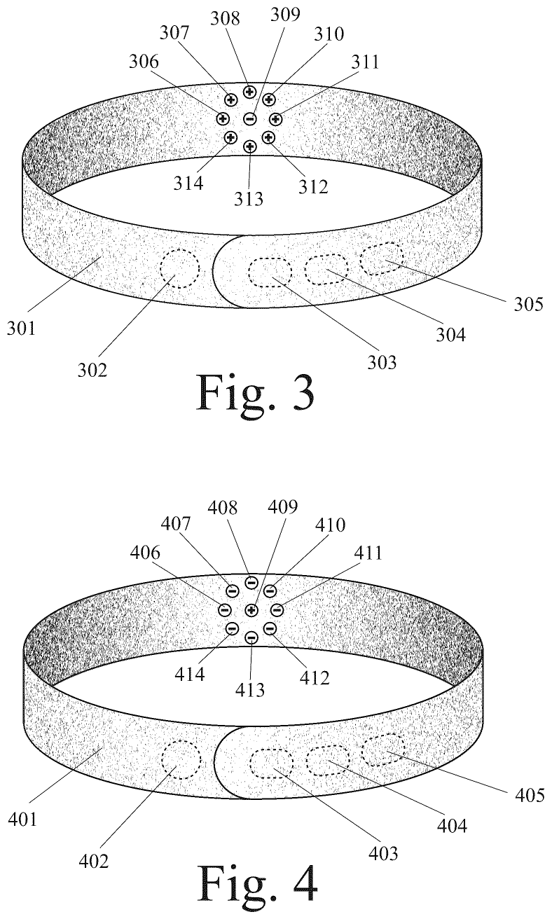

FIG. 3 shows an example of a wearable device with a light energy receiver and a plurality of light energy emitters around the light energy receiver. In this example, light energy emitters are configured in an octagonal array. In this example, the light energy emitters comprise the vertexes of an octagonal array. In an example, different light energy emitters can emit light energy at different times and/or in a chronological sequence.

Specifically, FIG. 3 shows an oblique-side-perspective view of an arcuate wearable device comprising: an arcuate band 301 which is configured to span (some or all of) the circumferential perimeter of a part of a person's body (such as a wrist, arm, finger, ankle, and/or leg); a plurality of light energy emitters 306, 307, 308, 310, 311, 312, 313, and 314 (identified with plus signs) which are configured to emit light energy toward the part of the person's body; a central light energy receiver 309 (identified with a negative sign) which is configured to receive light energy from the light energy emitters after that light energy has passed through and/or been reflected from the part of the person's body, wherein the plurality of light energy emitters is configured in an approximately-circular polygonal array around the central light energy receiver; a data processor 304 which receives data from the light energy receiver; an energy source 303 which provides energy to the light energy emitter and/or to the data processor; and a data transmitter 305 which transmits data from the data processor to a remote device and/or remote location. This example further comprises another (type of) biometric or environmental sensor 302.

In an example, a wearable device can collect data from an annular array of light emitters and receivers. In an example, a sensor can comprise a ring of light emitters and receivers. In an example, a sensor can comprise a polygonal array with at least one light emitter and at least one light receiver. In an example, data from biometric sensors can be analyzed to measure a person's oxygen level. In an example, a ring of biometric sensors can collect data which is used to measure a person's blood and/or tissue oxygenation.

In an example, a wearable device can comprise a circular, annular, and/or octagonal array of light emitters and/or light receivers. In an example, a wearable device (e.g. a smart watch or band) can comprise a circular, annular, and/or octagonal array of alternating light emitters and light receivers. In an example, a device can have an annular array of four light emitters. In an example, a device can have an annular array of four light receivers. In an example, a device can have an annular array of four light emitters. and four light receivers. In an example, light emitters in an annular array can have polar coordinates which differ by 60 or 90 degrees. In an example, light receivers in an annular array can have polar coordinates which differ by 60 or 90 degrees.

In an example, a wearable device can comprise an array of sets of light emitters and light receivers. In an example, different sets can be activated at different times. In an example, there can be multiple triads of energy emitters and energy receivers. In an example, a wearable ring of biosensors can comprise at least three light emitters which emit light energy of different colors. In an example, a wearable ring of biosensors can comprise at least three light emitters which emit light energy at different wavelengths. In an example, one light emitter can emit red light, a second light emitter can emit infrared light, and a third light emitter can emit green light.

In example, a wearable device can comprise: a housing that is attached to a wearable arcuate band; and a plurality of energy emitters and central energy receiver which are held by this housing. In an example, energy emitters can encircle the energy receiver. In an example, energy emitters can be arranged in an (approximately-circular) polygonal array around the central energy receiver. In an example, the housing and band can comprise a smart watch, fitness band, or wearable hydration monitor. In an example, the energy emitters and energy receiver can be located on the inward-facing (body-facing) side of a housing. In an example, the opposite (outward-facing) side of a housing can comprise a computer display and/or screen.

In an example, a central energy receiver can be centrally located with respect to the energy emitters. In an example, there can be eight energy emitters around the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

In example, a wearable device can comprise: an arcuate band which is configured to span (some or all of) the circumferential perimeter of a part of a person's body (such as a wrist, arm, finger, ankle, and/or leg); a housing which is attached to the arcuate band; a plurality of energy emitters which are held by the housing and configured to emit energy toward the part of the person's body; a central energy receiver which is held by the housing and configured to receive energy from the energy emitters, wherein the plurality of energy emitters are around the central energy receiver (in an approximately-circular polygonal array); a data processor which is held by the housing and receives data from the energy receiver which is analyzed in order to measure the person's body hydration level; an energy source which is held by the housing and provides energy to the energy emitters and/or to the data processor; and a data transmitter which is held by the housing and transmits data from the data processor to a remote device and/or remote location.

In an example, a wearable device can be a smart watch housing, smart watch band, or other type of wearable band which is used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level

In an example, a wearable device can comprise an (approximately-circular) polygonal array of three sets of red, infrared, and green light emitters and three light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of three sets of red, infrared, and green light emitters and three light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level. Other example variations and component elaborations discussed elsewhere in this disclosure (or in other disclosures within the priority-linked family) can also be applied to this example.

FIG. 4 shows an example of a wearable device (e.g. smart watch or band) with a light energy emitter and a plurality of light energy receivers around the light energy emitter. In this example, light energy receivers are in an octagonal array. In this example, the light energy receivers comprise vertexes of an octagonal array.

Specifically, FIG. 4 shows an oblique-side-perspective view of an arcuate wearable device comprising: an arcuate band 401 which is configured to span (some or all of) the circumferential perimeter of a part of a person's body (such as a wrist, arm, finger, ankle, and/or leg); a central light energy emitter 409 (identified with a plus sign) which is configured to emit light energy toward the part of the person's body; a plurality of light energy receivers 406, 407, 408, 410, 411, 412, 413, and 414 (identified with negative signs) which are configured to receive light energy from the light energy emitter after that light energy has passed through and/or been reflected from the part of the person's body, wherein the plurality of light energy receivers is configured in an approximately-circular polygonal array around the central light energy emitter; a data processor 404 which receives data from the light energy receivers; an energy source 403 which provides energy to the light energy emitter and/or to the data processor; and a data transmitter 405 which transmits data from the data processor to a remote device and/or remote location. This example further comprises another (type of) biometric or environmental sensor 402.

In an example, a wearable device can collect data from an annular array of light emitters and receivers. In an example, a sensor can comprise a ring of light emitters and receivers. In an example, a sensor can comprise a polygonal array with at least one light emitter and at least one light receiver. In an example, data from biometric sensors can be analyzed to measure a person's oxygen level. In an example, a ring of biometric sensors can collect data which is used to measure a person's blood and/or tissue oxygenation.

In an example, a wearable device can comprise a circular, annular, and/or octagonal array of light emitters and/or light receivers. In an example, a wearable device (e.g. a smart watch or band) can comprise a circular, annular, and/or octagonal array of alternating light emitters and light receivers. In an example, a device can have an annular array of four light emitters. In an example, a device can have an annular array of four light receivers. In an example, a device can have an annular array of four light emitters. and four light receivers. In an example, light emitters in an annular array can have polar coordinates which differ by 60 or 90 degrees. In an example, light receivers in an annular array can have polar coordinates which differ by 60 or 90 degrees.

In an example, a wearable device can comprise an array of sets of light emitters and light receivers. In an example, different sets can be activated at different times. In an example, there can be multiple triads of energy emitters and energy receivers. In an example, a wearable ring of biosensors can comprise at least three light emitters which emit light energy of different colors. In an example, a wearable ring of biosensors can comprise at least three light emitters which emit light energy at different wavelengths. In an example, one light emitter can emit red light, a second light emitter can emit infrared light, and a third light emitter can emit green light.

In example, a wearable device can comprise: a housing that is attached to a wearable arcuate band; and a plurality of energy emitters and central energy receiver which are held by this housing. In an example, energy emitters can encircle the energy receiver. In an example, energy emitters can be arranged in an (approximately-circular) polygonal array around the central energy receiver. In an example, the housing and band can comprise a smart watch, fitness band, or wearable hydration monitor. In an example, the energy emitters and energy receiver can be located on the inward-facing (body-facing) side of a housing. In an example, the opposite (outward-facing) side of a housing can comprise a computer display and/or screen.

In an example, a central energy receiver can be centrally located with respect to the energy emitters. In an example, there can be eight energy emitters around the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

In example, a wearable device can comprise: an arcuate band which is configured to span (some or all of) the circumferential perimeter of a part of a person's body (such as a wrist, arm, finger, ankle, and/or leg); a housing which is attached to the arcuate band; a plurality of energy emitters which are held by the housing and configured to emit energy toward the part of the person's body; a central energy receiver which is held by the housing and configured to receive energy from the energy emitters, wherein the plurality of energy emitters are around the central energy receiver (in an approximately-circular polygonal array); a data processor which is held by the housing and receives data from the energy receiver which is analyzed in order to measure the person's body hydration level; an energy source which is held by the housing and provides energy to the energy emitters and/or to the data processor; and a data transmitter which is held by the housing and transmits data from the data processor to a remote device and/or remote location.

In an example, a wearable device can be a smart watch housing, smart watch band, or other type of wearable band which is used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level

In an example, a wearable device can comprise an (approximately-circular) polygonal array of three sets of red, infrared, and green light emitters and three light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of three sets of red, infrared, and green light emitters and three light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level. Other example variations and component elaborations discussed elsewhere in this disclosure (or in other disclosures within the priority-linked family) can also be applied to this example.

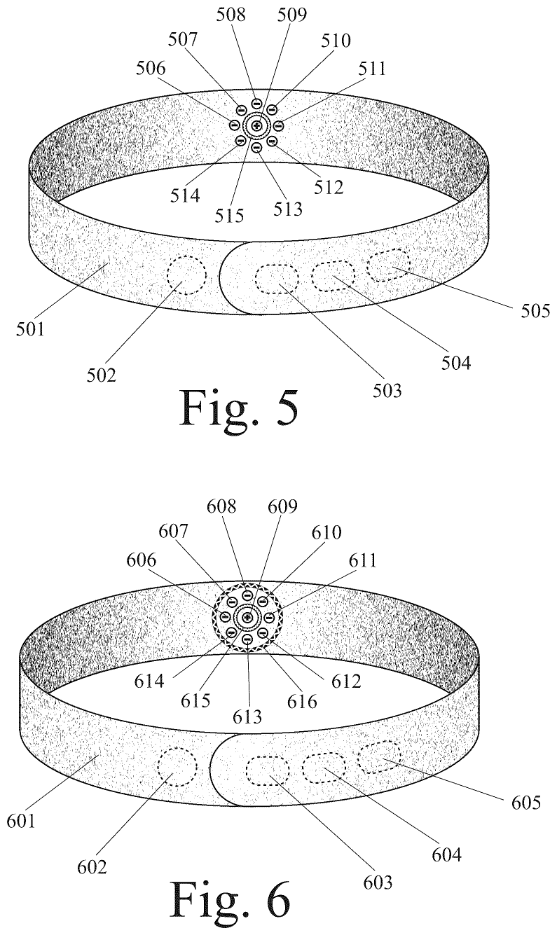

FIG. 5 shows an example of a wearable device with a single central light energy emitter, a plurality of light energy receivers around the central light energy emitter, and an light energy barrier between the central light energy emitter and the plurality of light energy receivers. In this example, light energy receivers are configured in an approximately-circular polygonal array around the central light energy emitter. In this example, the light energy barrier is arcuate. In this example, the light energy barrier is circular. In an example, the light energy barrier can be opaque. In an example wherein the type of light energy that is emitted and received is light energy, the light energy barrier can be made from a compressible material (such as foam).

Specifically, FIG. 5 shows an oblique-side-perspective view of a wearable device comprising: an arcuate band 501 which is configured to span (some or all of) the circumferential perimeter of a part of a person's body (such as a wrist, arm, finger, ankle, and/or leg); a central light energy emitter 509 (identified with a plus sign) which is configured to emit light energy toward the part of the person's body; a plurality of light energy receivers 506, 507, 508, 510, 511, 512, 513, and 514 (identified with negative signs) which are configured to receive light energy from the light energy emitter after that light energy has passed through and/or been reflected from the part of the person's body, wherein the plurality of light energy receivers is configured in an approximately-circular polygonal array around the central light energy emitter; an light energy barrier 515 between the central light energy emitter and the plurality of light energy receivers; a data processor 504 which receives data from the light energy receivers; an energy source 503 which provides energy to the light energy emitter and/or to the data processor; and a data transmitter 505 which transmits data from the data processor to a remote device and/or remote location. This example further comprises another (type of) biometric or environmental sensor 502.

In an example, a wearable device can collect data from an annular array of light emitters and receivers. In an example, a sensor can comprise a ring of light emitters and receivers. In an example, a sensor can comprise a polygonal array with at least one light emitter and at least one light receiver. In an example, data from biometric sensors can be analyzed to measure a person's oxygen level. In an example, a ring of biometric sensors can collect data which is used to measure a person's blood and/or tissue oxygenation.

In an example, a wearable device can comprise a circular, annular, and/or octagonal array of light emitters and/or light receivers. In an example, a wearable device (e.g. a smart watch or band) can comprise a circular, annular, and/or octagonal array of alternating light emitters and light receivers. In an example, a device can have an annular array of four light emitters. In an example, a device can have an annular array of four light receivers. In an example, a device can have an annular array of four light emitters. and four light receivers. In an example, light emitters in an annular array can have polar coordinates which differ by 60 or 90 degrees. In an example, light receivers in an annular array can have polar coordinates which differ by 60 or 90 degrees.

In an example, a wearable device can comprise an array of sets of light emitters and light receivers. In an example, different sets can be activated at different times. In an example, there can be multiple triads of energy emitters and energy receivers. In an example, a wearable ring of biosensors can comprise at least three light emitters which emit light energy of different colors. In an example, a wearable ring of biosensors can comprise at least three light emitters which emit light energy at different wavelengths. In an example, one light emitter can emit red light, a second light emitter can emit infrared light, and a third light emitter can emit green light.

In example, a wearable device can comprise: a housing that is attached to a wearable arcuate band; and a plurality of energy emitters and central energy receiver which are held by this housing. In an example, energy emitters can encircle the energy receiver. In an example, energy emitters can be arranged in an (approximately-circular) polygonal array around the central energy receiver. In an example, the housing and band can comprise a smart watch, fitness band, or wearable hydration monitor. In an example, the energy emitters and energy receiver can be located on the inward-facing (body-facing) side of a housing. In an example, the opposite (outward-facing) side of a housing can comprise a computer display and/or screen.

In an example, a central energy receiver can be centrally located with respect to the energy emitters. In an example, there can be eight energy emitters around the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

In example, a wearable device can comprise: an arcuate band which is configured to span (some or all of) the circumferential perimeter of a part of a person's body (such as a wrist, arm, finger, ankle, and/or leg); a housing which is attached to the arcuate band; a plurality of energy emitters which are held by the housing and configured to emit energy toward the part of the person's body; a central energy receiver which is held by the housing and configured to receive energy from the energy emitters, wherein the plurality of energy emitters are around the central energy receiver (in an approximately-circular polygonal array); a data processor which is held by the housing and receives data from the energy receiver which is analyzed in order to measure the person's body hydration level; an energy source which is held by the housing and provides energy to the energy emitters and/or to the data processor; and a data transmitter which is held by the housing and transmits data from the data processor to a remote device and/or remote location.

In an example, a wearable device can be a smart watch housing, smart watch band, or other type of wearable band which is used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level

In an example, a wearable device can comprise an (approximately-circular) polygonal array of three sets of red, infrared, and green light emitters and three light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of three sets of red, infrared, and green light emitters and three light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level. Other example variations and component elaborations discussed elsewhere in this disclosure (or in other disclosures within the priority-linked family) can also be applied to this example.

FIG. 6 shows an example of a wearable device with a single central light energy emitter, a plurality of light energy receivers configured around the central light energy emitter, a first light energy barrier between the central light energy emitter and the plurality of light energy receivers, and a second light energy barrier around the plurality of light energy receivers. In this example, light energy receivers are configured in an approximately-circular polygonal array around the central light energy emitter. In this example, the light energy barriers are arcuate. In this example, the light energy barriers are circular. In an example, the light energy barriers can be opaque. In an example wherein the type of light energy that is emitted and received is light energy, the light energy barriers can be made from a compressible material (such as foam).

Specifically, FIG. 6 shows an oblique-side-perspective view of a wearable device comprising: an arcuate band 601 which is configured to span (some or all of) the circumferential perimeter of a part of a person's body (such as a wrist, arm, finger, ankle, and/or leg); a central light energy emitter 609 (identified with a plus sign) which is configured to emit light energy toward the part of the person's body; a plurality of light energy receivers 606, 607, 608, 610, 611, 612, 613, and 614 (identified with negative signs) which are configured to receive light energy from the light energy emitter after that light energy has passed through and/or been reflected from the part of the person's body, wherein the plurality of light energy receivers is configured in an approximately-circular polygonal array around the central light energy emitter; a first light energy barrier 615 between the central light energy emitter and the plurality of light energy receivers; a second light energy barrier 616 around the plurality of light energy receivers; a data processor 604 which receives data from the light energy receivers; an energy source 603 which provides energy to the light energy emitter and/or to the data processor; and a data transmitter 605 which transmits data from the data processor to a remote device and/or remote location. This example further comprises another (type of) biometric or environmental sensor 602.

In an example, a wearable device can collect data from an annular array of light emitters and receivers. In an example, a sensor can comprise a ring of light emitters and receivers. In an example, a sensor can comprise a polygonal array with at least one light emitter and at least one light receiver. In an example, data from biometric sensors can be analyzed to measure a person's oxygen level. In an example, a ring of biometric sensors can collect data which is used to measure a person's blood and/or tissue oxygenation.

In an example, a wearable device can comprise a circular, annular, and/or octagonal array of light emitters and/or light receivers. In an example, a wearable device (e.g. a smart watch or band) can comprise a circular, annular, and/or octagonal array of alternating light emitters and light receivers. In an example, a device can have an annular array of four light emitters. In an example, a device can have an annular array of four light receivers. In an example, a device can have an annular array of four light emitters. and four light receivers. In an example, light emitters in an annular array can have polar coordinates which differ by 60 or 90 degrees. In an example, light receivers in an annular array can have polar coordinates which differ by 60 or 90 degrees.

In an example, a wearable device can comprise an array of sets of light emitters and light receivers. In an example, different sets can be activated at different times. In an example, there can be multiple triads of energy emitters and energy receivers. In an example, a wearable ring of biosensors can comprise at least three light emitters which emit light energy of different colors. In an example, a wearable ring of biosensors can comprise at least three light emitters which emit light energy at different wavelengths. In an example, one light emitter can emit red light, a second light emitter can emit infrared light, and a third light emitter can emit green light.

In example, a wearable device can comprise: a housing that is attached to a wearable arcuate band; and a plurality of energy emitters and central energy receiver which are held by this housing. In an example, energy emitters can encircle the energy receiver. In an example, energy emitters can be arranged in an (approximately-circular) polygonal array around the central energy receiver. In an example, the housing and band can comprise a smart watch, fitness band, or wearable hydration monitor. In an example, the energy emitters and energy receiver can be located on the inward-facing (body-facing) side of a housing. In an example, the opposite (outward-facing) side of a housing can comprise a computer display and/or screen.

In an example, a central energy receiver can be centrally located with respect to the energy emitters. In an example, there can be eight energy emitters around the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

In example, a wearable device can comprise: an arcuate band which is configured to span (some or all of) the circumferential perimeter of a part of a person's body (such as a wrist, arm, finger, ankle, and/or leg); a housing which is attached to the arcuate band; a plurality of energy emitters which are held by the housing and configured to emit energy toward the part of the person's body; a central energy receiver which is held by the housing and configured to receive energy from the energy emitters, wherein the plurality of energy emitters are around the central energy receiver (in an approximately-circular polygonal array); a data processor which is held by the housing and receives data from the energy receiver which is analyzed in order to measure the person's body hydration level; an energy source which is held by the housing and provides energy to the energy emitters and/or to the data processor; and a data transmitter which is held by the housing and transmits data from the data processor to a remote device and/or remote location.

In an example, a wearable device can be a smart watch housing, smart watch band, or other type of wearable band which is used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an annular and/or circular alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an annular and/or circular alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an approximately-circular (polygonal, such as quadrilateral, hexagonal, or octagonal) alternating (e.g. emitter then receiver) array of light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an approximately-circular (polygonal, such as quadrilateral, hexagonal, or octagonal) alternating (e.g. emitter then receiver) array of light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an array of alternating sets of red, infrared, and green light emitters and light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an array of alternating sets of red, infrared, and green light emitters and light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level

In an example, a wearable device can comprise an (approximately-circular) polygonal array of three sets of red, infrared, and green light emitters and three light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of three sets of red, infrared, and green light emitters and three light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of three sets of red, infrared, and green light emitters and three light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing side of the housing of a smart watch which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a watch band which are used to measure one or more of person's biometric parameters selected from the group consisting of: blood oxygenation level, body hydration level, heart rate, heart rate variability, blood pressure, and/or blood glucose level.

In an example, a wearable device can comprise an (approximately-circular) polygonal array of four sets of red, infrared, and green light emitters and four light receivers on the body-facing surface of the circumference of a fitness band which are used to measure one or more of person's biometric parameters selected from the group consisting of: blood oxygenation level, body hydration level, heart rate, heart rate variability, blood pressure, and/or blood glucose level. In an example, a wearable device can comprise an (approximately-circular) polygonal array of four sets of red, infrared, and green light emitters and four light receivers on the body-facing surface of the circumference of a smart finger ring which are used to measure one or more of person's biometric parameters selected from the group consisting of: blood oxygenation level, body hydration level, heart rate, heart rate variability, blood pressure, and/or blood glucose level. Other example variations and component elaborations discussed elsewhere in this disclosure (or in other disclosures within the priority-linked family) can also be applied to this example.

I claim:

1. A wearable device comprising:
a housing which is configured to be worn on a person's wrist, arm, or finger;
an array of light emitter sets and light receivers on the housing; wherein light emitted from the light emitters is reflected from and/or transmitted through the person's body tissue and received by the light receivers; wherein attributes of the light received by the light receivers are analyzed to measure one or more biometric parameters of the person; wherein there is an alternating pattern of light emitter sets and light receivers in the array; and wherein at least one light emitter in a set emits red light, at least one light emitter in the set emits infrared or near-infrared light, and at least one light emitter in the set emits green light;
a data processor; and
one or more opaque light barriers surrounding one or more light receivers, wherein the light barriers are compressible, compliant, and/or elastomeric.

2. A wearable device comprising:
a housing which is configured to be worn on a person's wrist, arm, or finger; and
an array of light emitter sets and light receivers on the housing, wherein light emitted from the light emitters is reflected from and/or transmitted through the person's body tissue and received by the light receivers, and wherein attributes of the light received by the light receivers are analyzed to measure one or more biometric parameters of the person.

3. The device in claim 2 wherein the device is a smart watch or wrist band.

4. The device in claim 2 wherein the device is a finger ring.

5. The device in claim 2 wherein the array is a circular, annular, or circumferential array.

6. The device in claim 2 wherein the array is a polygonal array.

7. The device in claim 6 wherein the array is a hexagonal or octagonal array.

8. The device in claim 2 wherein there is an alternating pattern of light emitter sets and light receivers in the array.

9. The device in claim 2 wherein there are three or more light emitters in each light emitter set.

10. The device in claim 9 wherein different light emitters in a set emit light with different colors, wavelengths, and/or frequencies, respectively.

11. The device in claim 10 wherein at least one light emitter in a set emits red light, at least one light emitter in the set emits infrared or near-infrared light, and at least one light emitter in the set emits green light.

12. The device in claim 2 wherein the attributes of the light which are analyzed include spectral distribution of the light.

13. The device in claim 2 wherein the device further comprises a data processor.

14. The device in claim 2 wherein the device further comprises one or more opaque light barriers surrounding one or more light receivers.

15. The device in claim 14 wherein the light barriers are compressible, compliant, and/or elastomeric.

16. The device in claim 14 wherein the light barriers reduce or eliminate transmission of light from the light emitters to the light receivers which has not been reflected by or transmitted through the person's body tissue.

17. The device in claim 14 wherein the light barriers reduce or eliminate transmission of ambient light to the light emitters.

18. The device in claim 14 wherein each light barrier surrounds a single light receiver.

19. The device in claim 14 wherein a single light barrier surrounds all of the light receivers.

20. A wearable device comprising:
an arcuate band which is configured to span a person's wrist, arm, finger;
a housing which is attached to the arcuate band;
a plurality of light energy emitters which are held by the housing and configured to emit light energy toward the person's body;
a central light energy receiver which is held by the housing and configured to receive light energy from the light energy emitters, wherein the plurality of light energy emitters are around the central light energy receiver in an approximately-circular polygonal array;
a data processor which is held by the housing and receives data from the light energy receiver; and
a data transmitter which is held by the housing and transmits data from the data processor to a remote device and/or remote location.
